(19) United States
(12) Patent Application Publication
(10) Pub. No.: US 2015/0031655 A1
(43) Pub. Date: Jan. 29, 2015

(54) COMBINATION OF LIVER X RECEPTOR MODULATOR AND ESTROGEN RECEPTOR MODULATOR FOR THE TREATMENT OF AGE-RELATED DISEASES

(71) Applicant: Othman Ghribi, Grand Forks, ND (US)

(72) Inventor: Othman Ghribi, Grand Forks, ND (US)

(73) Assignee: UNIVERSITY OF NORTH DAKOTA, Grand Forks, ND (US)

(21) Appl. No.: 14/111,916

(22) PCT Filed: Apr. 10, 2012
(86) PCT No.: PCT/US12/32924

§ 371 Date: Sep. 24, 2014

Related U.S. Application Data
(60) Provisional application No. 61/476,035, filed on Apr. 15, 2011.

(51) Int. Cl. A61K 31/575; A61K 31/341; A61K 31/4035; A61K 31/566; A61K 31/05; A61K 31/46; A61K 31/4045; A61K 31/428; A61K 31/4985; A61K 31/4468; A61K 31/496; A61K 31/5513; A61K 31/195; A61K 31/352

(52) U.S. Cl. CPC A61K 31/575; A61K 31/195; A61K 31/341; A61K 31/4035; A61K 31/566; A61K 31/05; A61K 31/352; A61K 31/4045; A61K 31/428; A61K 31/4985; A61K 31/4468; A61K 31/496; A61K 31/5513; A61K 31/46
USPC 514/171; 514/567; 514/471; 514/417; 514/456; 514/418; 514/367; 514/250; 514/329; 514/253.07; 514/220; 514/304

(57) ABSTRACT

The disclosure provides a method of treating a mammal afflicted with an age-related disorder, comprising administering to the mammal a combination of liver X receptor (LXR) modulator and estrogen receptor (ER) modulator, in an amount effective to treat the mammal. Further disclosed are the LXR modulators and ER modulators used in the combination therapy.

### CROSS-REFERENCE TO RELATED APPLICATIONS

This application claims priority from U.S. provisional patent application No. 61/476,035, filed Apr. 15, 2011.

### GOVERNMENT FUNDING

This invention was made with the support of the NIH/NIEHS under grant no. RO1ES014826. The Government has certain rights in this invention.

### BACKGROUND OF THE INVENTION

As our population ages, clinicians will see increasing numbers of patients who seek medical attention with memory or other cognitive complaints, as well as other diseases or disorders of aging, such as vision loss and movement disorders.

The most common dementia causes are Alzheimer's disease (AD), vascular dementia, Lewy body disease and frontotemporal dementia. Although these seem to exist independently of each other, postmortem autopsy of those diagnosed with dementia in the community typically show mixed, rather than unitary, brain abnormalities (e.g., presence of both plaques and tangles as well as microinfarcts). See, e.g., J. A. Schneider et al., Neurology, 60, 2187 (2007). There is general agreement that Alzheimer's disease is the most prevalent cause of dementia, followed by vascular dementia (VaD), and then either frontotemporal dementia (FTD) or Lewy body disease (LBD). Less common causes of dementia include normal-pressure hydrocephalus (NPH), traumatic brain injury (TBI), acquired immune deficiency syndrome (AIDS), Huntington disease (HD), prion diseases (e.g., Creutzfeldt-Jakob disease), primary progressive aphasia (PPA), corticobasal degeneration (CBD), and dementia of depression (in the past referred to as pseudodementia).

Parkinson's disease (PD) is the most common movement disorder. Most of the current available drugs for PD can at best manage the symptoms associated with this disease and their long-term use is associated with significant motor and cognitive impairments. The vast majority of PD cases are sporadic, and genetic forms account for only a small percentage of this disorder. The exact causes of sporadic PD are not known but environmental factors, dietary agents and genetic susceptibility are suspected to participate in the pathogenesis of this disease. Identification of factors and cellular mechanisms by which these factors cause PD will help in designing efficacious therapies that can prevent, reverse or stop the progression of this disease.

Although the metabolic pathways that enhance or inhibit the progression of age-related dementia, movement disorders and vision loss, e.g., due to macular degeneration, are largely unknown, recent research has focused on the role of oxysterols. These compounds are oxidative metabolites of cholesterol and their production can be enhanced by increased levels of the oxidative stress sensor heme oxygenase-1 (HO-1). The oxysterol 27-hydroxycholesterol (27-OHC) simultaneously reduces tyrosine hydroxylase (TH), the rate limiting enzyme in dopamine synthesis, and increases α-synuclein levels. Dementia with Lewy bodies is also associated with α-synuclein accumulation.

In epidemiological studies related to Alzheimer's disease, 27-OHC was found to inhibit expression of the adipocytokine leptin, an effect which may correlate with increased levels of amyloid-β and p-tau, and the onset of AD.

Finally, oxidative stress and inflammation, possibly mediated by dietary cholesterol and 27-OHC, are believed to play a key role in the pathogenesis of age-related macular degeneration (AMD), but no effective pharmaceutical treatment for AMD has been developed.

Therefore, a continuing need exists to explicit the biochemical pathways responsible for the development of such age-related disorders, in order to identify targets for screening possible interventional drug therapies, as well as to identify agents that effectively treat these conditions, or block their onset.

### SUMMARY OF THE INVENTION

The present invention provides for a method of treating a mammal afflicted with an age-related disorder such as a dementing condition, a movement or vision disorder or symptoms thereof. The method includes administering to the mammal a combination of at least one liver X receptor (LXR) modulator, such as an LXR antagonist, and at least one estrogen receptor (ER) modulator, such as an ER agonist, in an amount effective to treat the mammal, e.g., to inhibit, reduce or ameliorate at least one symptom of the disorder. An outline of the underlying mechanisms of action operative in the present method is given in FIG. 8, wherein 27-OHC is 27-hydroxycholesterol, TH is tyrosine hydroxylase and DA is dopamine.

In a specific embodiment, the mammal is a human. In a more specific embodiment, the mammal is a human of at least about 50 years old in age.

In a specific embodiment, the age-related disorder includes age-related macular degeneration (AMD), Parkinson's disease (PD), dementia with Lewy bodies (DLB), synucleinopathies, dyskinesia, (including bradykinesia, akinesia and dystonia), Alzheimer's disease (AD), dementia, multiple system atrophy (MSA) including Shy-Drager syndrome, pure autonomic failure (PAF), or Pick disease (PiD).

In another specific embodiment, the age-related disorder includes a cognitive dysfunction selected from the group consisting of dementia, age-related deficit in cognitive performance, stress-related deficit in cognitive performance, mild cognitive impairment (MCI), schizophrenia, Alzheimer's disease (AD), and symptoms associated thereof.

In another specific embodiment, the age-related disorder includes dementia selected from the group consisting of vascular dementia (VaD), dementia of the Alzheimer's type, dementia due to HIV disease, dementia due to head trauma, dementia due to Parkinson's disease (PD), dementia due to Huntington's disease, dementia due to Pick's disease, dementia due to Creutzfeldt-Jacob disease, substance-induced persisting dementia, dementia due to multiple etiologies, dementia with Lewy bodies (DLB), ischemia/stroke, tangles, and global dementia.

In another specific embodiment, the age-related disorder includes dementia of the Alzheimer's type selected from the group consisting of dementia of the Alzheimer's type without behavioral disturbance, dementia of the Alzheimer's type with behavior disturbance, dementia of the Alzheimer's type with early onset, and/or dementia of the Alzheimer's type with late onset.

In another specific embodiment, the age-related disorder includes dry age-related macular degeneration (AMD) or wet age-related macular degeneration (AMD).

In one specific embodiment, the liver X receptor (LXR) modulator is a liver X receptor (LXR) antagonist.

In another specific embodiment, the liver X receptor (LXR) modulator is an α liver X receptor (LXRα) antagonist or is a β liver X receptor (LXRβ) antagonist.

In another specific embodiment, the liver X receptor (LXR) modulator is an α liver X receptor (LXRα) modulator, or is a β liver X receptor (LXRβ) modulator.

In another specific embodiment, the liver X receptor (LXR) modulator includes at least one of:

5α,6α-epoxycholesterol-3-sulfate (ECHS);

3-[3-[N-(2-Chloro-3-trifluoromethylbenzyl)-(2,2-diphenylethyl)amino]propyloxy]phenylacetic acid hydrochloride (GW3965);

ketocholesterol-3-sulfate;

2,4,6-Trimethyl-N-{[3′-(methylsulfonyl)-4-biphenylyl]methyl}-N-{[5-(trifluoromethyl)-2-furanyl]methyl}benzenesulfonamide (G-SK2033); or

5-choloro-N-2′-n-pentylphenyl-1,3-dithiophthalimide (5CPPSS).

In one specific embodiment, the estrogen receptor (ER) modulator is an α estrogen receptor (ERα) modulator, or a β estrogen receptor (ERβ) modulator.

In another specific embodiment, the estrogen receptor (ER) modulator is an α estrogen receptor (ERα) agonist, or a β estrogen receptor (ERβ) agonist.

In another specific embodiment, the estrogen receptor (ER) modulator includes:

17β-estradiol (E2), Fulvestrant (ICI182780); Stilphostrol® (diethylstilbesterol diphosphate); or

Daidzein (7-Hydroxy-3-(4-hydroxyphenyl) chromen-4-one).

In one specific embodiment, the treatment further includes administering to the mammal at least one of a heme oxygenase, e.g., HO-1, inhibitor, dopamine receptor agonist, dopamine precursor, monoamine oxidase B (MAO-B) inhibitor, catechol-O-methyltransferase (COMT) inhibitor, additional dopaminergic agent, anti-cholinergic, cholinesterase inhibitor, N-methyl-D-aspartic acid or N-methyl-D-aspartate (NMDA) receptor antagonist, and/or an anti-psychotic (anti-depressant).

In another specific embodiment, the treatment further includes administering to the mammal the active pharmaceutical ingredient (API) of at least one of Requip® (ropinirole), Mirapex® (pramipexole), Parlodel® (bromocriptine), Apokyn® (apomorpine), Sinemet® (levodopa-carbidopa), Eldepryl® (selegiline-deprenyl), Emsam® (selegiline), Azilect® (rasagiline), Tasmar® (tolcapone), Comtan® (entacapone), Symmetrel® (amantadine), Artane® (trihexyphenidyl), Cogentin® (benzatropine), Aricept® (donepezil), Exelon® (rivastigmine), Namenda® (memantine), Clozaril® (clozapine), Abilify® (aripiprazole), and/or Zelapar® (selegiline hydrochloride), CERE-120, ACP-103 or SR57667B; wherein the active pharmaceutical ingredient (API) exists as the free acid, free base, or pharmaceutically acceptable salt.

In one specific embodiment, the composition includes a pharmaceutically acceptable carrier or diluents.

In one specific embodiment, the administration is oral, parenteral, intravenous (i.v.), intraocular, intravitreal or intraperitoneal (i.p.) or stereotactic neurosurgical intracranial injection.

In one specific embodiment, the liver X receptor (LXR) modulator is administered in at least about 5 mg/day.

In another specific embodiment, the liver X receptor (LXR) modulator is administered once per day (q.d.), or twice a day (b.i.d.).

In one specific embodiment, the estrogen receptor (ER) modulator is administered in at least about 5 mg/day.

In another specific embodiment, the treatment further includes the estrogen receptor (ER) modulator and is administered once per day (q.d.), or twice a day (b.i.d.).

In one specific embodiment, the administration of the liver X receptor (LXR) modulator and the estrogen receptor (ER) modulator is approximately simultaneous or concurrent in time.

In another specific embodiment, the administration of the liver X receptor (LXR) modulator and the estrogen receptor (ER) modulator is consecutive in time.

In one specific embodiment, the liver X receptor (LXR) modulator and the estrogen receptor (ER) modulator are present within a single unit dosage form.

In one specific embodiment, the administration occurs for a period of time of at least about 4 weeks.

### BRIEF DESCRIPTION OF THE FIGURES

FIG. 1. Effects of 27-OHC and concomitant estradiol treatment on TH expression in SH-SY 5Y neuroblastoma cells. (a) Representative Western blot, (b) densitometric analysis, and (c) real time RT-PCR analysis demonstrate that 27-OHC decreases TH protein and mRNA levels. Co-treatment with estradiol precludes the attenuation imposed by 27-OHC on TH protein and mRNA levels. Estradiol increases the expression of TH protein and mRNA. Data is expressed as Mean+S.E.M and includes determinations made in four separate cell culture experiments (n=4). *p<0.05 and **p<0.01 versus control; †p<0.05 versus 27-OHC.

FIG. 2. Effects of 27-OHC and concomitant Estradiol treatment on ERα and ERβ translocation to the nucleus. (a) Representative Western blot and (b) densitometric analysis demonstrate that 27-OHC reduces the levels of ERα in the nucleus while concomitant estradiol treatment completely overrides the 27-OHC-induced inhibition in nuclear translocation of ERα. Estradiol also increases levels of ERα in the nucleus. (c) Representative Western blot and (d) densitometric analysis clearly demonstrate that 27-OHC reduces the nuclear translocation of ERβ to the nucleus. Co-treatment with estradiol completely reverses the 27-OHC-induced inhibition in nuclear translocation of ERβ. Estradiol also increases levels of ERβ in the nucleus. Data is expressed as Mean+S.E.M and includes determinations made in four separate cell culture experiments (n=4). *p<0.05 and **p<0.01 versus control; ††p<0.01 versus 27-OHC.

FIG. 3. Effects of 27-OHC and concomitant estradiol treatments on the binding of ERα and ERβ to the ERE in TH promoter. (a) EMSA shows that 27-OHC decreases the binding of ERα/β to the exogenous oligonucleotide probe. Treatment of cells with 27-OHC and estradiol or with estradiol alone significantly increases the binding of ERα/β to the exogenous oligonucleotide probe that corresponds to the ERE-half site in the TH promoter. (b) ChIP analysis demonstrates that there are no significant differences in ERα binding to the ERE-half site in the TH promoter in response to treatments with 27-OHC, estradiol, or both. Moreover, ChIP assay shows that in the basal state ERβ bound to the ERE-half site is 10-fold higher than ERα bound to the same ERE-half site in the TH promoter. 27-OHC significantly attenuates the binding of ERβ to the ERE-half site in the TH promoter. Treatment with estradiol alone or in combination with 27-OHC significantly increases the binding of ERβ to the ERE-half site in the TH promoter. Data is expressed as Mean+S.E.M and includes determinations made in four separate cell culture experiments (n=4). ***p<0.001 versus control; †††p<0.001 versus 27-OHC.

FIG. 4. (a) Dual-luciferase assay demonstrating the effect of 27-OHC on ER-mediated transcription. Cells were transfected with reporter construct comprising of ERE coupled upstream of the firefly luciferase gene. 27-OHC significantly attenuates the ERE-mediated transcription of luciferase gene. (b) Dual-luciferase assay demonstrating the effects of 27-OHC and concomitant estradiol treatments on the TH promoter activity. Cells were transfected with a reporter construct containing the TH promoter fused upstream of the firefly luciferase gene. 27-OHC significantly reduces TH promoter activity, while concomitant estradiol treatment completely precludes the inhibition imposed by 27-OHC on TH promoter activity. Data is expressed as Mean+S.E.M and includes determinations made in four separate cell culture experiments (n=4). ***p<0.001 versus control; †††p<0.001 versus 27-OHC.

FIG. 5. Effects of 27-OHC and concomitant treatment with LXR agonist GW3965 and LXR antagonist ECHS on α-synuclein expression in SH-SY 5Y neuroblastoma cells. (a) Representative Western blot (b) densitometric analysis, and (c) real time RT-PCR analysis demonstrate that 27-OHC significantly increases α-synuclein protein and mRNA levels. Treatment with the LXR agonist GW3965 also significantly increases α-synuclein protein and mRNA levels. Co-treatment with 27-OHC and the LXR agonist GW3965 accentuates the increase induced by 27-OHC on α-synuclein protein levels, but not mRNA levels. Treatment with the LXR antagonist ECHS produces no effect on the levels of α-synuclein protein and mRNA. However, the LXR antagonist ECHS significantly reverses the 27-OHC-induced increase in α-synuclein protein and mRNA levels. Data is expressed as Mean+S.E.M and includes determinations made in four separate cell culture experiments (n=4). **p<0.01and ***p<0.001 versus control; ††p<0.01 and †††p<0.001 versus 27-OHC; ΔΔp<0.01 versus ECHS.

FIG. 6. Effects of 27-OHC and concomitant treatment with LXR agonist GW3965 and LXR antagonist ECHS on LXRα and LXRβ levels in the nucleus. Representative Western blot and densitometric analysis demonstrate that 27-OHC increases levels of LXRα (a,b) and LXRβ (c,d) in the nucleus. Treatment with the LXR agonist GW3965 also significantly increases LXRα and LXRβ levels in the nucleus. Co-treatment with 27-OHC and the LXR agonist GW3965 increases the LXRα and LXRβ levels in the nucleus to the same extent as treatment with 27-OHC or GW3965 alone. Treatment with the LXR antagonist ECHS markedly reduces the nuclear levels of LXRα and LXRβ. ECHS also significantly reverses the 27-OHC-induced increase in the nuclear levels of LXRα and LXRβ. Data is expressed as Mean+S.E.M and includes determinations made in four separate cell culture experiments (n=4). *p<0.05, **p<0.01 and ***p<0.001 versus control; †p<0.05 and ††p<0.01 versus 27-OHC; ΔΔp<0.01 versus ECHS.

FIG. 7. Effects of 27-OHC and concomitant treatment with LXR agonist GW3965 and LXR antagonist ECHS on the binding of LXRα/β to the LXRE in the α-synuclein promoter. (a) EMSA was performed with a double stranded biotin labeled oligonucleotide probe (20 fmoles) corresponding to the LXRE sites in the α-synuclein promoter region—SITE1: 13788 nucleotides upstream of transcription start site (−13796 to −13767). The EMSA shows that LXR agonist GW3965 and 27-OHC increase binding of LXRα/β to the exogenous oligonucleotide probe corresponding to the α-synuclein promoter region. The LXR antagonist, on the other hand, significantly reduces the 27-OHC-induced increase in LXR binding to the oligonucleotide probe. (b) Chromatin Immunoprecipitation (ChIP) assay was performed by precipitating one quarter of the chromatin each with 5 μg of LXRα or LXRβ antibody. ChIP analysis demonstrates that there are no significant differences in LXRα binding to the LXRE in the α-synuclein promoter. However, ChIP assay shows that in the basal state LXRβ bound to the LXRE is 5.5-fold higher than LXRα bound to the same LXRE in the α-synuclein promoter. The LXR agonist GW3965 and 27-OHC significantly augment the binding of LXRβ to the LXRE in the α-synuclein promoter. The LXR antagonist ECHS significantly reduces the binding of LXRβ to the LXRE compared to control. Furthermore, ECHS significantly inhibits the 27-OHC-induced increase in binding of LXRβ to the LXRE in the α-synuclein promoter. Data is expressed as Mean+S.E.M and includes determinations made in four separate cell culture experiments (n=4). *p<0.05 and ***p<0.001 versus control; ††p<0.01 versus 27-OHC; ΔΔΔp<0.001 versus ECHS.

FIG. 8. A flowchart summarizing the mechanisms of action of the present invention.

FIG. 9 depicts the effects of a combination of estradiol (E2) and ECHS on TH expression levels in human neuroblastoma cells.

FIG. 10 depicts the effects of a combination of E2 and ECHS on α-synuclein expression levels in neuroblastoma cells.

FIG. 11 depicts the effects of a combination of E2, ECHS and 27-OHC on TH and α-synuclein expression levels.

FIG. 12. Treatment of ARPE-19 cells for 24 h with 27-OHC increases ROS (left panel) and TNF-α levels (right panel). Co-treatment with ECHS, but not E2, reduced reactive oxygen species (ROS) levels. Co-treatment with E2, but not ECHS, reduced increased TNF-α levels. *p<0.05 vs. control; ^{#}p<0.05 vs. 27-OHC. All treatments were applied at 10 μM concentrations.

### DETAILED DESCRIPTION

Reference will now be made in detail to certain claims of the present invention, examples of which are illustrated in the accompanying structures and formulas. While the present invention will be described in conjunction with the enumerated claims, it will be understood that the description below is not intended to limit the present invention. On the contrary, the present invention is intended to cover all alternatives, modifications, and equivalents, which may be included within the scope of the present invention as defined by the claims.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention relates to the use of a combination of a liver X receptor (LXR) modulator and an estrogen receptor (ER) modulator for the treatment of age-related diseases. When describing the combination of a liver X receptor (LXR) modulator and an estrogen receptor (ER) modulator for the treatment of age-related diseases, the following terms have the following meanings, unless otherwise indicated.

### DEFINITIONS

Unless stated otherwise, the following terms and phrases as used herein are intended to have the following meanings:

When tradenames are used herein, applicants intend to independently include the tradename product and the active pharmaceutical ingredient(s) of the tradename product.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The phrase “pharmaceutically acceptable salts” refers to ionic compounds wherein a parent non-ionic compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include conventional non-toxic salts and quaternary ammonium salts of the parent compound formed, for example, from non-toxic inorganic or organic acids. Non-toxic salts can include those derived from inorganic acids such as hydrochloric, hydrobromic, hydroiodic, sulfuric, sulfamic, phosphoric, nitric and the like. Salts prepared from organic acids can include those such as acetic, 2-acetoxybenzoic, ascorbic, benzenesulfonic, benzoic, citric, ethanesulfonic, ethane disulfonic, formic, fumaric, gentisinic, glucaronic, gluconic, glutamic, glycolic, hydroxymaleic, isethionic, isonicotinic, lactic, maleic, malic, methanesulfonic, oxalic, pamoic (1, F-methylene-bis-(2-hydroxy-3-naphthoate)), pantothenic, phenylacetic, propionic, salicylic, sulfanilic, toluenesulfonic, stearic, succinic, tartaric, bitartaric, and the like. Certain compounds can form pharmaceutically acceptable salts with various amino acids. For a review on pharmaceutically acceptable salts see Berge et al., J. Pharm. Sci. 1977, 66(1), 1-19, which is incorporated herein by reference.

The pharmaceutically acceptable salts of the compounds described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of many suitable salts are found in Remington's Pharmaceutical Sciences, 17th ed., Mack Publishing Company, Easton, Pa., (1985), 1418, and the disclosure of which is incorporated herein by reference.

“Therapeutically effective amount” is intended to include an amount of a combination of active compounds described herein, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g., prophylaxis); (ii) inhibiting the pathologic condition or arresting its development; (iii) relieving the pathologic condition; and/or (iv) diminishing symptoms associated with the pathologic condition.

As used herein, “antagonism” refers to the interference in the physiological action of a chemical substance by another having a similar structure. For instance, a receptor antagonist is an agent that reduces the response that a ligand produces when the receptor antagonist binds to a receptor on a cell. The antagonist can function either “directly” (by binding to the receptor to decrease its function) and/or “indirectly” (by decreasing the expression of the ligand).

As used herein, a “receptor antagonist” refers to a type of receptor ligand or drug that does not provoke a biological response itself upon binding to a receptor, but blocks or dampens agonist-mediated responses. In pharmacology, antagonists have affinity for, but no efficacy at their cognate receptors, and binding will disrupt the interaction and inhibit the function of an agonist or inverse agonist at receptors. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding. The majority of drug antagonists achieve their potency by competing with endogenous ligands or substrates at structurally-defined binding sites on receptors.

### Pharmaceutical Formulations

The combination of active compounds of this present invention is formulated with conventional carriers and excipients, which will be selected in accord with ordinary practice. Tablets will contain excipients, coatings, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients, 5^{th} Ed.; Rowe, Sheskey, and Owen, Eds.; American Pharmacists Association; Pharmaceutical Press: Washington, D.C., 2006. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the combination of active ingredients to be administered alone, it may be preferable to present them as pharmaceutical formulations. The formulations, both for veterinary and for human use, of the present invention comprise the combination of active ingredients, as described herein, together with one or more acceptable carriers therefor and optionally other therapeutic ingredients. The carrier(s) are typically “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences, Mack Publishing Company, Easton, Pa., (1985). Such methods include the step of bringing into association the combination of active ingredients with the carrier which constitutes one or more accessory ingredients. In general, the formulations are prepared by uniformly and intimately bringing into association the combination of active ingredients with liquid carriers or finely divided solid carriers or both, and then, if necessary, shaping the product.

Formulations of the present invention suitable for oral administration may be presented as discrete units such as capsules, cachets or tablets each containing a predetermined amount of the combination of active ingredients; as a powder or granules; as a solution or a suspension in an aqueous or non-aqueous liquid; or as an oil-in-water liquid emulsion or a water-in-oil liquid emulsion. The combination of active ingredients may also be administered as a bolus, electuary or paste.

A tablet is made by compression or molding, optionally with one or more accessory ingredients. Compressed tablets may be prepared by compressing in a suitable machine the combination of active ingredients in a free-flowing form such as a powder or granules, optionally mixed with a binder, lubricant, inert diluent, preservative, surface active or dispersing agent. Molded tablets may be made by molding in a suitable machine a mixture of the powdered combination of active ingredients moistened with an inert liquid diluent. The tablets may optionally be coated or scored and optionally are formulated so as to provide slow or controlled release of the combination of active ingredients therefrom.

For administration to the eye or other external tissues e.g., mouth and skin, the formulations are preferably applied as a topical ointment or cream containing the combination of active ingredients in an amount of, for example, 0.075 to 20% w/w (including active ingredients) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the combination of active ingredients may be employed with either a paraffinic or a water-miscible ointment base. Alternatively, the combination of active ingredients may be formulated in a cream with an oil-in-water cream base. In other embodiments, the combination of active ingredients may be formulated in a topical skin patch, e.g., a transdermal skin patch.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the combination of active ingredients through the skin or other affected areas. Examples of such dermal penetration enhancers include dimethyl sulphoxide and related analogs.

The oily phase of the emulsions of this present invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the present invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a combination of active ingredients (as described herein), together with one or more pharmaceutically acceptable carriers or excipients and optionally other therapeutic agents. Pharmaceutical formulations containing the combination of active ingredients may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the combination of active ingredients in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the combination of active ingredients is mixed with an inert solid diluent, for example calcium phosphate or kaolin, or as soft gelatin capsules wherein the combination of active ingredients is mixed with water or an oil medium, such as peanut oil, liquid paraffin or olive oil.

Aqueous suspensions of the present invention contain the combination of active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the combination of active ingredients in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the present invention suitable for preparation of an aqueous suspension by the addition of water provide the combination of active ingredients in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the present invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the present invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredients that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredients per milliliter of solution in order that infusion of a suitable volume at a rate of about 30 mL/hr can occur.

Formulations suitable for administration to the eye include eye drops wherein the combination of active ingredients is dissolved or suspended in a suitable carrier, especially an aqueous solvent for the active ingredient. The combination of active ingredients is preferably present in such formulations in a concentration of 0.5 to 20%, advantageously 0.5 to 10% particularly about 1.5% w/w.

Formulations suitable for topical administration in the mouth include lozenges comprising the combination of active ingredients in a flavored basis, usually sucrose and acacia or tragacanth; pastilles comprising the combination of active ingredients in an inert basis such as gelatin and glycerin, or sucrose and acacia; and mouthwashes comprising the combination of active ingredients in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of a given condition.

Formulations suitable for vaginal administration may be presented as pessaries, tampons, creams, gels, pastes, foams or spray formulations containing in addition to the combination of active ingredients such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this present invention may include other agents conventional in the art having regard to the type of formulation in question, for example those suitable for oral administration may include flavoring agents.

The present invention further provides veterinary compositions comprising at least one combination of active ingredients as above defined together with a veterinary carrier thereof.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compositions of the present invention can be formulated to provide an immediate release of the combination of active ingredients. Compositions of the present invention can also be formulated to provide controlled, extended or sustained release of the combination of active ingredients to allow less frequent dosing or to improve the pharmacokinetic or toxicity profile of the active ingredient. Accordingly, the present invention provides compositions formulated for immediate, controlled, extended or sustained release.

### Therapeutic Compositions

Compositions of the present invention can include agonists, modulators, antagonists, precursors, inhibitors and agents. For example, to treat an age related disorder, a caregiver can administer at least one liver X receptor modulator, at least one estrogen receptor (ER) modulator, or a combination of the two.

Liver X receptors (LXRs) include LXRα and LXRβ, and are ligand-activated transcription factors of the nuclear receptor superfamily that control the expression of genes involved in cholesterol and fatty acid metabolism. As used herein, “liver X receptor (LXR) modulator” refers to a substance or compound capable of adjusting or regulating the activity or degree of activity of the LXR. An LXR modulator can regulate the activity of the α or β isoform of LXR. As such, as used herein “α liver X receptor (LXR) modulator” refers to a compound that modulates the LXR isoform LXRα. As used herein “β liver X receptor (LXR) modulator” refers to a compound that modulator the LXR isoform LXRβ.

An LXR modulator can include a LXR antagonist. As used herein, “liver X receptor (LXR) antagonist” refers to a compound that reduces the action or activity of an LXR. Antagonism can occur directly or indirectly. In particular, LXR antagonists can exist in α or β form. As used herein “α liver X receptor (LXR) antagonist” refers to a compound that antagonizes the LXR isoform LXRα. Similarly, as used herein “β liver X receptor (LXR) antagonist” refers to a compound that antagonizes the LXR isoform LXRβ.

Estrogen receptors are a group of receptors that are activated by the hormone estrogen, estrogen derivatives or estrogenic substances. One type of estrogen receptor is the nuclear hormone receptor superfamily of intracellular receptors, which are DNA-binding transcription factors that regulate gene expression. There are two forms of the estrogen receptor, referred to as estrogen receptor α and estrogen receptor β. A modulator can bind to either form of the receptor and promote interactions with coactivators or corepressors. As used herein, “estrogen receptor (ER) modulator” refers to a compound that is capable of adjusting or regulating the activity or degree of activity of the ER. An estrogen receptor (ER) modulator can include an a estrogen receptor (ERα) modulator or a β estrogen receptor (ERβ) modulator. As used herein, “α estrogen receptor (ERα) modulator” refers to a compound that modulates ERα. As used herein, “β estrogen receptor (ERβ) modulator” refers to a compound that modulates ERβ.

An ER modulator can include an ER agonist. An agonist is a compound that binds a receptor and triggers a cellular response. As used herein, “α estrogen receptor (ERα) agonist” refers to a compound that binds to ERα and triggers a cellular response. As used herein, “β estrogen receptor (ERβ) agonist” refers to a compound that binds to ERβ and triggers a cellular response.

In another example, to treat an age related disorder, a caregiver can administer a dopamine precursor. Dopamine is a catecholamine neurotransmitter with multiple functions within the brain, including affecting an individual's movement and cognition. For example, insufficient dopamine biosynthesis has been postulated to cause Parkinson's disease. In another example, dopamine disorders have been shown to cause a decline in neurocognitive functions. Dopamine cannot be given as an effective drug to treat neurological disorders because dopamine does not cross the blood-brain barrier. Dopamine precursors help to solve this therapeutic problem. As used herein a “dopamine precursor” refers to a compound that can cross the blood-brain barrier and can subsequently be metabolized into dopamine. Dopamine receptor agonists have also been shown to have therapeutic value. As used herein a “dopamine receptor agonist” refers to a compound that binds the dopamine receptor and triggers a cellular response similar to that of dopamine. A “dopaminergic agent,” as used herein refers to an agent that stimulates dopamine receptors.

Another agent that can be used to treat an age related disorder is a catechol-O-methyltransferase (COMT) inhibitor. Catechol-O-methyl transferase (COMT) is an enzyme that can degrade catecholamines such as dopamine, epinephrine, and norepinephrine. Drugs inhibiting COMT can alter the availability of catecholamines. As used herein a “catechol-O-methyltransferase (COMT) inhibitor” refers to a compound that inhibits the COMT enzyme and results in an increased availability of a catecholamine.

Another agent that can be used to treat an age related disorder is an anticholinergic agent. As used herein an “anti-cholinergic” refers to an agent that blocks the neurotransmitter acetylcholine. In blocking acetylcholine, parasympathetic nerve impulses can be inhibited. In some cases, these agents can reduce spasms of smooth muscle. In an example, an anti-cholinergic agent can be used to treat Parkinson's disease. In another example, an antipsychotic drug can have anticholinergic effects.

Cholinesterase inhibitors can also help the nervous system of humans to function properly. As used herein a “cholinesterase inhibitor” refers to an agent that binds to the enzyme cholinesterase and does not allow it to biodegrade acetylcholine. For example, cholinesterase inhibitors have been used to treat Alzheimer's disease, dementia, delirium and traumatic brain injuries.

N-methyl-D-aspartic acid (NMDA) class of glutamate receptors have been implicated as a molecular device for controlling memory function. The NMDA receptor forms a heterotetramer and serves a non-specific cation channel that plays a key role in memory processes. As used herein an “N-methyl-D-aspartic acid antagonist” refers to an agent that binds to the NMDA acid and reduces its activity. As used herein an “N-methyl-D-aspartate (NMDA) receptor antagonist” refers to an agent that binds to the NMDA receptor and reduces the activity of the receptor. In an example, a NMDA antagonist can be used to treat age-related diseases. Examples of age-related diseases can include Alzheimer's disease and schizophrenia.

Depression is a symptom that can accompany an age related disorder. To treat depression, an anti-psychotic compound can be administered. As used herein, an “anti-psychotic (anti-depressant)” refers to a compound that is primarily used to manage psychosis. Likewise, an antidepressant is a compound used to alleviate mood disorders including depression and anxiety. One such type of antidepressant can include a monoamine oxidase inhibitor (MAOI). As used herein a “monoamine oxidase inhibitor” or “MAOI” refers to a compound used to primarily treat depression, which acts by inhibiting the activity of monoamine oxidase, thereby preventing the breakdown of monoamine neurotransmitters and increasing their availability. The physiological result can include an increased amount of serotonin, melatonin, epinephrine and norepinephrine and dopamine. The “monoamine oxidase inhibitor” or “MAOI” can include a monoamine oxidase A (MAO A) inhibitor, a monoamine oxidase B (MAO B) inhibitor, or a combination thereof

### Dosages

An effective dose of a combination of active ingredients depends at least on the nature of the condition being treated, toxicity, whether the compound is being used prophylactically (lower doses), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses of a given agent.

### Diseases and Disorders

As used herein, “dementia” is an umbrella term for several symptoms related to a decline in cognitive skills. Dementia refers to a general mental deterioration due to physiological or psychological factors; characterized by disorientation, impaired memory, judgment, and intellect, and a shallow labile effect. Common symptoms include a gradual loss of memory, problems with reasoning or judgment, disorientation, difficulty in learning, loss of language skills, and decline in the ability to perform routine tasks. People with dementia typically experience changes in their personalities and behavioral problems, such as agitation, anxiety, delusions (believing in a reality that does not exist), and hallucinations (seeing, feeling or hearing things that do not exist). Dementia can be focal or global. Focal dementias are dementias where the problem cluster is limited or localized to a specific area(s) of the brain. “Global dementias” are dementias where the problem cluster and damage are located in several areas of the brain.

Specific types of dementia include, e.g., vascular dementia (VaD), dementia of the Alzheimer's type, dementia due to HIV disease, dementia due to head trauma, dementia due to Parkinson's disease, dementia due to Huntington's disease, dementia due to Pick's disease, dementia due to Creutzfeldt-Jacob disease, substance-induced persisting dementia, dementia due to multiple etiologies, and global dementia. See, e.g., Diagnostic and Statistical Manual of Mental Disorders, 4^{th} edition, Text Revision (DSM-IV-TR) (2000).

As used herein, “dementia of the Alzheimer's type,” “Alzheimer's disease,” or “AD” refers to a general mental deterioration due to physiological or psychological factors; characterized by disorientation, impaired memory, judgment, and intellect, and a shallow labile effect. See, e.g., Stedman's Medical Dictionary, 11^{th} edition (1990). Alzheimer's disease (AD) is a progressive, degenerative disease of the brain and is one of several disorders that cause the gradual loss of brain cells. Specific types of dementia of the Alzheimer's type include, e.g., dementia of the Alzheimer's type without behavioral disturbance, dementia of the Alzheimer's type with behavior disturbance, dementia of the Alzheimer's type with early onset, and dementia of the Alzheimer's type with late onset. See, e.g., Diagnostic and Statistical Manual of Mental Disorders, 4^{th} edition, Text Revision (DSM-IV-TR) (2000). As used herein, “dementia of the Alzheimer's type with behavioral disturbance” refers to dementia of the Alzheimer's type occurring with an impairment such as wandering, getting lost, repetitive phenomena, sleep disturbance, hyperphagia, hoarding behavior, aggression, agitation, incontinence, and poor personal hygiene. As used herein, “dementia of the Alzheimer's type without behavioral disturbance” refers to dementia of the Alzheimer's type that is not accompanied by significant behavioral problems.

Alzheimer's disease is sometimes referred to as an age-related disorder. As used herein, “age-related disorder” refers to a disorder or disease that is increasingly prevalent in an elderly population. Alzheimer's disease is most common in the elderly, but can be diagnosed at a variety of ages. For example, Alzheimer's disease can be an early or late onset disease. As used herein, “dementia of the Alzheimer's type with early onset” refers to dementia of the Alzheimer's type that is diagnosed before the age of 65. In some cases, dementia of the Alzheimer's type with early onset can develop in people in their 30's. In other cases, dementia of the Alzheimer's type with early onset can develop in people in their 40's. As used herein, “dementia of the Alzheimer's type with late” refers to dementia of the Alzheimer's type that is diagnosed after the age of 65, and is the most common form of Alzheimer's disease.

Alzheimer's Disease (AD) and age related macular degeneration both involve the formation of β-amyloid-containing plaques and tangles which are also thought to contribute to dementia in some instances. “Plaques” (also referred to as “amyloid plaques”) as used herein, refers to pathologic accumulations of β-amyloid protein fragments often found in aging brains, which are considered as the major pathology of Alzheimer's disease and similar to “drusen deposits” found in age-related macular degeneration (AMD). “Tangles” (alternatively referred to as “neurofibrillary tangles” or “taupathies”) as used herein, refers to insoluble, aberrant cytoplasmic filaments or inclusions that are often found inside the brain cells of individuals afflicted with dementia. The tangles contain hyperphosphorylated and self-assembled tau proteins which destabilize microtubules to affect normal structure and function of the cells. The term “synucleinopathies,” as used herein, refers to a group of neurodegenerative disorders characterized by fibrillary aggregates of proteins in neurons and glia. Examples of these disorders include Parkinson's disease, dementia with Lewy bodies, pure autonomic failure, and multiple system atrophy. Some of these disorders will be discussed further below.

As used herein, “vascular dementia,” or “VaD” refers to a general mental deterioration most frequently occurring subsequent to a decrease in blood flow to parts of the brain, occurring due to vascular events, such as an ischemia/stroke or vascular lesion. The term “ischemia/stroke,” as used herein, refers to a restriction in blood supply, particularly a blood supply to the brain, accompanied by resultant damage. Vascular dementia is commonly characterized by disorientation, experiencing memory deficits, inattentiveness, apathy, psychosis, delusions, hallucinations, paranoia, severe depression, and mood and behavioral changes. Vascular dementia is the second most common form of dementia after Alzheimer disease. In some instances, the condition is not a single disease, but is a group of syndromes relating to various vascular mechanisms. Subtypes of vascular dementia can include a mild vascular cognitive impairment, multi-infarct dementia, vascular dementia due to a strategic single infarct, vascular dementia due to lacunar lesions, vascular dementia due to hemorrhagic lesions, Biswanger disease, subcortical vascular dementia, mixed dementia (combination of Alzheimer's disease and vascular dementia), cortical dementia, and subcortical dementia.

As used herein, “dementia due to HIV disease” refers to a general cognitive impairment associated with HIV; characterized by cognitive deficits, behavioral changes and impaired motor involvement. Symptoms can include cognitive changes such as slowed processing of information, behavioral abnormalities, unsteady gait, tremor, or weakness. As used herein, “dementia due to HIV disease” can include AIDS dementia complex (ADC), HIV-associated dementia (HAD), minor cognitive motor disorder (MCMD), asymptomatic neurocognitive impairment (ANI), and HIV-associated mild neurocognitive disorder (MND).

As used herein, “dementia due to head trauma” refers to the presence of a dementia that is deemed to be the direct pathophysiological consequence of head trauma; often characterized by posttraumatic amnesia or memory impairment. The symptoms can vary depending upon the location and extent of the brain injury, but can include aphasia, attentional problems, irritability, anxiety, depression or affective lability, apathy, increased aggression, or other changes in personality. Dementia due to head trauma is typically nonprogressive, but repeated head injury can lead to a progressive dementia.

As used herein, “dementia due to Parkinson's disease,” refers to the presence of dementia that is judged to be the direct pathophysiological consequence of Parkinson's disease. Briefly, Parkinson's disease is a slowly progressive neurological condition characterized by tremor, rigidity, bradykinesia, and postural instability. As used herein, “Parkinson's disease (PD)” refers to a degenerative disorder of the central nervous system often characterized by the accumulation of proteins in neurons and loss of dopamine in the striatum due to progressive degeneration of the midbrain dopaminergic neurons. Dementia due to Parkinson's disease is frequently characterized by cognitive and motoric slowing, executive dysfunction and impairment in memory retrieval. In some cases additional syndromes manifest themselves with dementia, including progressive supranuclear palsy, olivopontocerebellar degeneration, and vascular dementia.

At autopsy, some individuals with Parkinson's are found to have coexisting neuropathology indicative of Alzheimer's disease or of diffuse Lewy body disease, including loss of dopaminergic neurons and the accumulation of α-synuclein. “Dementia with Lewy bodies (DLB),” as used herein refers to dementia caused by or occurring with Lewy bodies in the brain. Lewy bodies are abnormal protein aggregates inside neurons comprising primarily of α-synuclein, and the formation of Lewy bodies leads to neural degeneration.

The causes of PD are likely multi-factorial with several factors including environmental agents and genetic susceptibility participating in the pathogenesis of the disease. Most of the animal models for sporadic PD are formulated through the administrations of neurotoxins, such as 6-hydroxydopamine, rotenone and MPTP. These models reproduce some of the pathological aspects of PD, in particular decreased TH immuno-reactivity and accumulation of α-synuclein. However, the cellular mechanisms that regulate α-synuclein accumulation and TH reduction induced by neurotoxins or in humans with PD are still to be identified.

Maintenance of dopamine synthesis and reduction in α-synuclein accumulation may represent a potential neuroprotective strategy against AD. Recently, it was demonstrated that the oxysterol 27-hydroxycholesterol (27-OHC) simultaneously reduces the expression levels of tyrosine hydroxylase (TH), the rate-limiting enzyme in dopamine synthesis, and increases production of α-synuclein in human neuroblastoma cells (R. Antham Prabhakara et al., J. Neurochem., 107, 1722 (2008)). This study also showed that 24-hydroxycholesterol (24-OHC) can reverse the 27-OHC-induced effects on α-synuclein and TH. However, the cellular mechansims involved in 27-OHC effects on TH and α-synuclein have not been determined.

As used herein, “dementia due to Huntington's disease,” refers to the presence of dementia that is judged to be the direct pathophysiological consequence of Huntington's disease. Briefly, Huntington's disease is a progressive degenerative disease affecting cognition, emotion and movement. Symptoms of dementia due to Huntington's disease commonly include mood disorders, aggressiveness, psychosis, and psychiatric disorders such as mania, eccentricity, inappropriateness, loss of social amenities, excess irritability, depression, apathy, and social withdrawal.

As used herein, “dementia due to Pick's disease,” refers to the presence of dementia that is judged to be the direct pathophysiological consequence of Pick's disease. Briefly, Pick's disease is a degenerative brain disease affecting the frontal and/or temporal lobes. As used herein, “Pick disease (PiD)” refers to the clinical syndrome known as frontotemporal lobar degeneration. Dementia due to Pick's disease is frequently clinically characterized by changes in personality, deterioration of social skills, emotional blunting, behavioral disinhibition, prominent language abnormalities, difficulties with memory, apraxia, apathy, and extreme agitation. Clinically, dementia due to Pick's disease can be difficult to distinguish with certainty from atypical cases of Alzheimer's disease or other dementias that affect the frontal lobe.

As used herein, “dementia due to Creutzfeldt-Jacob disease,” refers to the presence of dementia that is judged to the direct pathophysiological consequence of Creutzfeldt-Jacob disease. Briefly, Creutzfeldt-Jacob disease is one of the subacute spongiform encephalopathies, which is a group of central nervous system diseases caused by transmissible agents known as “slow viruses” or prions. Symptoms of dementia due to Creutzfeldt-Jacob disease can include mood swings, memory problems, lack of interest, loss of memory, personality changes, depression, and hallucinations. Clinically, Creutzfeldt-Jacob disease can be difficult to diagnose in a living person, as the most certain way of confirming the presence of Creutzfeldt-Jacob disease is to examine brain tissue at autopsy.

As used herein, “substance-induced persisting dementia,” refers to the presence of dementia that is judged to be a result of substance use or substance abuse. Substances that can contribute to substance-induced persisting dementia can include, but are not limited to: alcohol, inhalants, sedatives, hypnotics, and anxiolytics. Substance-induced persisting dementia is frequently characterized by memory impairment, aphasia, apraxia, agnosia and disturbance in executive functioning. The deficits do not occur exclusively during the course of substance use induced delirium and persist beyond the usual duration of substance abuse related withdrawal.

As used herein, “dementia due to multiple etiologies,” refers to a dementia that has more than one etiology. For example, more than one medical condition may be etiologically related to a dementia, such as dementia of the Alzheimer's type and dementia due to head trauma. In another example, a dementia can be due to the combined effects of a general medical condition, such as Parkinson's disease and long-term substance abuse.

As discussed above, symptoms of dementia include various signs of cognitive dysfunction. As used herein, “cognitive dysfunction” refers to a general condition characterized by poor mental function associated with confusion, forgetfulness, and difficulty concentrating. As used herein, “mild cognitive impairment (MCI)” refers to mild forgetfulness, language or another mental function that may be noticeable by others, but is not severe enough to interfere with daily life. An MCI may worsen with age. As used herein, “age-related deficit in cognitive performance” refers to a decline in cognitive performance or function that progressively worsens with age. Cognitive function can also be influenced by stress. As used herein, “stress-related deficit in cognitive performance” refers to deficits in cognitive performance related to or in response to stress.

Also discussed above, symptoms of dementia can include mental health problems. For example, dementia can be accompanied by schizophrenia. As used herein, “schizophrenia” refers to a condition or mental disorder characterized by a disintegration of thought processes or emotional responsiveness. The disorder commonly manifests itself as hallucinations, paranoid delusions, disorganized thoughts and speech, and is often accompanied by social dysfunction.

As discussed above, dementia can be a symptom of or accompanied by movement disorders. As used herein, “dyskinesia” refers to a movement disorder characterized by diminished or absence of voluntary movement (hypokinesia) or the presence of involuntary movement (hyperkinesia). Dyskinesia can also be characterized by gait impairment and/or postural instability. As used herein, “bradykinesia” refers to a movement disorder characterized by the slowed ability to begin a movement or continue a movement. As used herein, “akinesia” refers to the loss and/or absence of movement.

Another age-related disorder is age-related macular degeneration (AMD). AMD is a condition characterized by degeneration of the macula, which is part of the retina responsible for central vision. AMD is also often associated with the presence of β-amyloid containing “drusen deposits.” As used herein, “age-related macular degeneration (AMD)” refers to degeneration of the macula occurring in patients older than 50. AMD can be diagnosed as wet or dry. As used herein, “dry age-related macular degeneration (AMD)” refers to non-neovascular AMD, which is macular degeneration not accompanied by an abnormal growth of new blood vessels in an eye. As used herein, “wet age-related macular degeneration (AMD)” refers to neovascular AMD, which is macular degeneration that is accompanied by an abnormal growth of new blood vessels in an eye.

Another age-related disorder is multiple system atrophy (MSA). As used herein, “multiple system atrophy (MSA) including Shy-Drager syndrome” refers to a rare neurological disorder that impairs the body's involuntary functions including, but not limited to, blood pressure, heart rate, bladder function, and digestion. Symptoms can be similar to those seen in patients with Parkinson's disease, such as slowness of movement, muscle rigidity and poor balance. MSA frequently develops in adulthood, such as in individuals older than 50.

Another age-related disorder is pure autonomic failure (PAF). As used herein, “pure autonomic failure (PAF)” refers to a degenerative disease of the autonomic nervous system frequently characterized by significantly reduced levels of catecholamines. Symptoms typically include unsteadiness, dizziness, or faintness upon standing, pain in the neck or back of the head, a loss of ability to sweat, and changes in urination.

### Routes of Administration

Compositions of the present invention are administered by any route appropriate to the condition to be treated. Suitable routes include oral, or parenteral, including rectal, nasal, topical (including buccal and sublingual), vaginal and by subcutaneous, intraperitoneal (i.p.) intramuscular, intravenous, intradermal, intraocular, intravitreal, intrathecal and epidural infusion or injection or stereotactic neurosurgical intracranial injection, and the like. Administration via implanted pumps or depots as well as transdermal administration via films, bandages or patches is also useful in some cases. It will be appreciated that the preferred route may vary with for example the condition of the recipient.

### Combination Therapy

The combination of liver X receptor (LXR) modulator and estrogen receptor (ER) modulator can also be used alone, or in combination with other active ingredients. In either embodiment, the specific combination is selected based on the condition to be treated, cross-reactivities of ingredients and pharmaco-properties of the combination.

The combination of liver X receptor (LXR) modulator and estrogen receptor (ER) modulator can also be used alone, or in combination with other active ingredients, in a unitary dosage form for simultaneous or sequential administration to a patient. In either embodiment, the combination therapy may be administered as a simultaneous or sequential regimen. When administered sequentially, the combination may be administered in two or more administrations.

The combination therapy may provide “synergy” and “synergistic effect,” i.e., the effect achieved when the active ingredients used together is greater than the sum of the additive effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. In either case, an effective therapeutic amount of each agent is present in vivo simultaneously.

Pharmaceutical kits useful in the present invention, which include a therapeutically effective amount of a pharmaceutical composition that includes (a) a liver X receptor (LXR) modulator and (b) an estrogen receptor (ER) modulator, in one or more sterile containers, are also within the ambit of the present invention. Sterilization of the container may be carried out using conventional sterilization methodology well known to those skilled in the art. Component (a) and component (b) may be in the same sterile container or in separate sterile containers. The sterile containers or materials may include separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

The invention will be further described by reference to the following detailed examples.

### I. Introduction

Emerging data suggests that estradiol regulates TH expression in vivo (N. R. Thanky et al., Brain Res. Molec. Brain Res., 104, 220 (2002)) and in vitro (J. Maharjan et al., J. Neurochem., 112, 42 (2010)) by modulating TH promoter activity via estrogen receptor (ER) signaling. Estradiol is the most prominent endogenous estrogen and an agonist of the estrogen receptor α (ERα) and estrogen receptor β (ERβ). The TH promoter contains an estrogen response element (ERE) half site to which activated estrogen receptors (ERα and ERβ) can bind and induce TH promoter activity (L. Serova et al., Neuroendocrin., 75, 193 (2002)).

The signal transduction mechanisms involved in α-synuclein expression are not well known. The involvement of LXR signaling pathway in the regulation of α-synuclein expression has been recently suggested (D. Cheng et al., Neuroreport, 19, 1685 (2008)). However, it is not clear whether LXRα or LXRβ is involved in the regulation of α-synuclein expression.

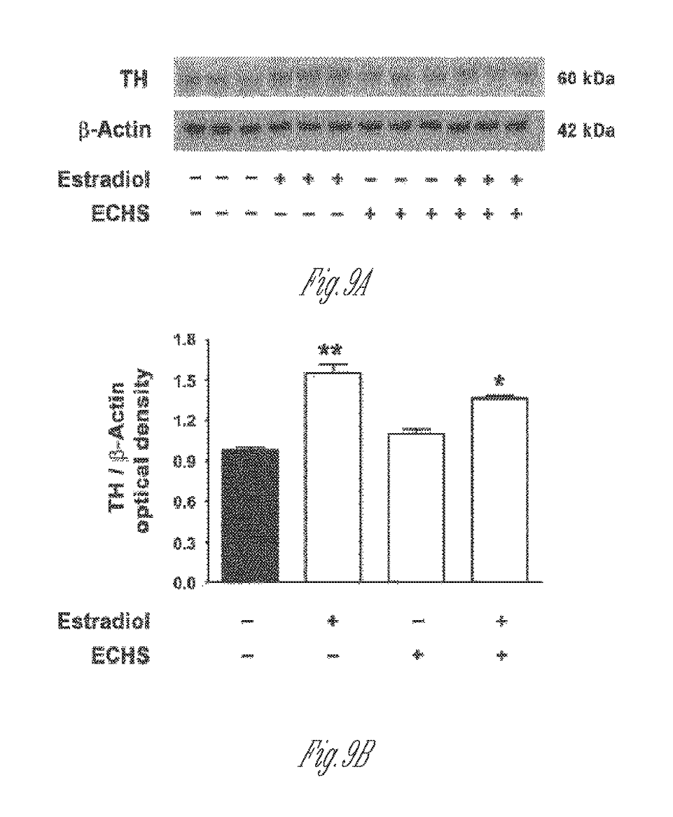

The following examples will explore the role of ER and LXR in the regulation of TH and α-synuclein expression and characterize the extent to which the oxysterol 27-OHC utilizes these pathways to modulate expression levels of these two proteins. The results show that inhibition of LXRβ reduces α-synuclein expression while activation of ERβ increases TH expression, which can reduce synuclein accumulation. See FIG. 8.

### II. Experimental Procedures

### A. Materials

27-OHC was purchased from Medical Isotopes (Pelham, N.H.). Estradiol (E2) and the LXR agonist GW3965 were purchased from Sigma Aldrich (Saint Louis, Mo.). The LXR antagonist 5α-6α-epoxycholesterol-3-sulfate (ECHS) was purchased from Steraloids Inc. (Newport, R.I.). ERE-Luciferase (ERE reporter monitors the activity of estrogen receptor induced signal transduction pathways; a mixture of inducible ERE-responsive firefly luciferase construct and constitutively expressing Renilla luciferase construct) and LXRE-Luciferase (Cignal LXR Reporter (luc) kit: Catalog # CCS-0041L; the LXR reporter measures the transcriptional activity of liver X receptor (LXR)) DNA constructs were purchased from SA Biosciences (Frederick, Md.). The human TH-Luciferase promoter construct was purchased from SwitchGear Genomics (Menlo Park, Calif.). All cell culture reagents, with the exception of fetal bovine serum (Atlanta Biologicals, Lawrenceville, Ga.) and antibiotic/antimycotic mix (Sigma Aldrich, Saint Louis, Mo.) were purchased from Invitrogen (Carlsbad, Calif.). Human SH-SY5Y neuroblastoma cells were purchased from ATCC (Manassas, Va.).

### B. Cell Culture and Treatments

Human neuroblastoma SH-SY5Y cells were grown in DMEM/F12 medium containing 10% fetal bovine serum (FBS) and 1% antibiotic/antimycotic mix. Cells were maintained at 37° C. in a saturated humidity atmosphere containing 95% air and 5% CO₂. After reaching 80% confluence, cells were incubated with vehicle (control), 10 μM 27-OHC, 1 nM estradiol, 10 μM 27-OHC+1 nM estradiol, 10 μM GW3965, 10 μM 27-OHC+10 μM GW3965, 10 μM ECHS, and 10 μM 27-OHC+10 μM ECHS for 24 hours at 37° C. in cell medium.

### C. Luciferase Reporter Assays

Constructs encoding ERE and TH promoter conjugated to the firefly luciferase gene were used in the study. Human neuroblastoma SH-SY5Y cells were plated in 96-well plates at a density of 2×10⁴ cells/well.

The cells were transfected when 80% confluent with 0.25 μg of either ERE-firefly Luciferase reporter construct, LXRE-firefly Luciferase reporter construct, or TH promotor-firefly Luciferase promoter construct. Respective non-inducible reporter constructs containing constitutively expressing Renilla luciferase were used as negative internal controls. Constitutively expressing GFP constructs were used as positive control to determine transfection efficiency.

Cells were incubated for 24 hours with Opti-MEM serum free medium (Invitrogen, Carlsbad, Calif.) containing the reporter constructs dissolved in transfection reagent. After 24 hours the medium was changed and the cells were incubated in normal DMEM/F12 medium containing 10% FBS and cells were treated with the different treatment regimens.

The cells were treated in triplicate and harvested 24 hours later and subjected to dual-luciferases assay. The dual-luciferase assay was performed using a “Dual-Luciferase Reporter Assay System” from Promega (Madison, Wis.). The luminescence recorded is expressed as Relative Luminescence Units (RLU) and normalized to per mg protein. Unit value was assigned to control and the magnitude of differences among the samples is expressed relative to the unit value of control cells.

### D. Western Blot Analysis

Treated SH-SY5Y cells were washed with PBS and trypsinized to collect the cells and centrifuged at 5000 g. The pellet was washed again with PBS and homogenized in NE-PER tissue protein extraction reagent (Thermo Scientific, Rockford, Ill.) supplemented with protease and phosphatase inhibitors. Protein concentrations from the cytosolic and nuclear homogenates were determined with BCA protein assay.

Proteins (10 μg) were separated on SDS-PAGE gels, transferred to a polyvinylidene difluoride membrane (BioRad, Hercules, Calif.), and incubated with the following monoclonal antibodies: anti-TH mouse antibody (1:1000; Sigma Aldrich, Saint Louis, Mo.), anti-α-synuclein mouse antibody (1:500; Chemicon, Temecula, Calif.), anti-ERα rabbit antibody (1:500; Abcam, Cambridge, Mass.), anti-ERβ rabbit antibody (1:200; Upstate, Lake Placid, N.Y.), anti-LXRα rabbit antibody (1:500; Abcam, Cambridge, Mass.), anti-LXRβ mouse antibody (1:500; Abcam, Cambridge, Mass.).

β-actin and Lamin A were used as a gel loading control for cytosolic homogenates and nuclear homogenates respectively. The blots were developed with enhanced chemiluminescence (Immmun-star HRP chemiluminescent kit, Bio-Rad, Hercules, Calif.). Bands were visualized on a polyvinylidene difluoride membrane and analyzed by LabWorks 4.5 software on a UVP Bioimaging System (Upland, Calif.). Quantification of results was performed by densitometry and the results analyzed as total integrated densitometric values (arbitrary units).

### E. Quantitative Real Time RT-PCR Analysis

Total RNA was isolated and extracted from treated cells using the 5 prime “PerfectPure RNA tissue kit” (5 Prime, Inc., Gaithersburg, Md.). RNA estimation was performed using “Quant-iT RNA Assay Kit” using a Qubit fluorometer according to the manufacturer's protocol (Invitrogen, Carlsbad, Calif.). cDNA was synthesized by reverse transcribing 1 μg of extracted RNA using an “iScript cDNA synthesis kit” (BioRad, Hercules, Calif.). The oligomeric primers (Sigma, St Louis, Mo.) used to amplify the TH mRNA and α-synuclein mRNA are enumerated in Table 1. The cDNA amplification was performed using an iQ SYBR Green Supermix kit following the manufacturer's instructions (BioRad, Hercules, Calif.). The amplification was performed using an iCycler iQ Multicolor Real Time PCR Detection System (BioRad, Hercules, Calif.). The expression of specific TH and α-synuclein transcripts amplified were normalized to the expression of glyceraldehyde-3-phosphate dehydrogenase (GAPDH).

### F. Electrophoretic Mobility Shift Assay (EMSA)

The Electrophoretic Mobility Shift Assay (EMSA) was performed using a kit from Active Motif (Carlsbad, Calif.) following manufacturer's protocol. Nuclear extract was prepared using NE-PER protein extraction reagent following the manufacturer's instructions (Thermo Scientific, Rockford, Ill.).

The 5′-biotin labeled and unlabeled oligonucleotide probes that correspond to the ERE binding site (−51 to −20 of the TH promoter) in the TH promoter region and LXRE binding sites (−13796 to −13767 of the α-synuclein promoter) in the α-synuclein promoter region (Table 1) were purchased from Sigma Aldrich (St Louis, Mo.).

TABLE 1
Primers designed and used for TH, α-synuclein,
TH promoter and α-synuclein promoter

| | | GenBank | | |
|---|---|---|---|---|
| | | Accession | | |
| GENE | PRIMER | Number | SEQUENCE | APPLICATION |
| TH | Forward | NM 199292 | actggttcacggtggagttc | RT-PCR |
| | | | (SEQ ID NO: 1) | |
| TH | Reverse | NM 199292 | agctcctgagcttgtccttg | RT-PCR |
| | | | (SEQ ID NO: 2) | |
| α- | Forward | NM 000345 | tgtgcccagtcatgacattt | RT-PCR |
| synuclein | | | (SEQ ID NO: 3) | |
| α- | Reverse | NM 000345 | ccacaaaatccacagcaca | RT-PCR |
| synuclein | | | (SEQ ID NO: 4) | |
| TH | Site-1 | NM 199292 | gctttgacgtcagctcagct | EMSA |
| promoter | | | tataagaggctgctgggcc | |
| | | | a (SEQ ID NO: 5) | |
| α- | Site-1 | NT 016354 | aagggaagcagatcataaa | EMSA |
| synuclein | | | agttcagaaaa (SEQ | |
| promoter | | | ID NO: 6) | |
| TH | Forward | NM 199292 | ctccatcaggcacagcag | ChIP |
| promoter | | | (SEQ ID NO: 7) | |
| TH | Reverse | NM 199292 | ggcccagcagcctcttat | ChIP |
| promoter | | | (SEQ ID NO: 8) | |
| α- | Forward | NT 016354 | caagatgtgacttgggtgct | ChIP |
| synuclein | | | (SEQ ID NO: 9) | |
| promoter | | | | |
| α- | Reverse | NT 016354 | tgggattttgttttctatcaca | ChIP |
| synuclein | | | (SEQ ID NO: 10) | |
| promoter | | | | |

Ten μg of nuclear proteins were incubated with either 20 femtomoles of biotin labeled oligonucleotide probes or 4 picomoles of unlabeled oligonucleotide probes. To exhibit specificity of the biotin labeled oligonucleotide probes, unlabeled oligonucleotide probes were used as specific competitors for binding reactions at 200-fold greater concentration than that of the biotin labeled probes. 1 μg of Poly d(I-C) was used as a non-specific competitor for binding reactions. The resulting binding reaction mix was loaded and resolved on a 5% TBE gel (Bio Rad, Hercules, Calif.) followed by transfer onto a nylon membrane. The bands were visualized using the HRP-Streptavidin-Chemiluminescent reaction mix provided with the kit on a UVP Bioimaging System (Upland, Calif.).

### G. Chromatin Immunoprecipitation (ChIP) Analysis

ChIP analysis was performed to evaluate the extent of ERα/ERβ and LXRα/LXRβ binding to the DNA elements in the TH promoter and α-synuclein promoter regions respectively using “SimpleChIP™ Enzymatic Chromatic IP kit” from Cell Signaling (Boston, Mass.).

Briefly, cells from each treatment group (3×10⁶ cells) were washed with PBS, trypsinized, centrifuged at 5000 g. The pellet containing the cells was further washed with PBS and cross-linked using 37% formaldehyde for 15 min followed by the addition of glycine solution to cease the cross-linking reaction.

The cells were washed with 4× volumes of 1×PBS and centrifuged at ˜220 g for 5 min. The pellet was resuspended and incubated for 10 min in 5 ml of tissue lysis buffer containing DTT and protease and phosphatase inhibitors. The subsequent steps to isolate the cross-linked chromatin were performed according to the manufacturer's protocol.

The cross-linked chromatin from each sample was apportioned into four equal parts. One quarter of the cross-linked chromatin was set aside as “input.” One quarter of the cross-linked chromatin from each sample was incubated with 5 μg of anti-ERα mouse antibody (Cell Signaling, Boston, Mass.) and 5 μg of anti-ERβ rabbit antibody (Upstate, Bedford, Mass.), or 5 μg of anti-LXRα rabbit antibody (Abcam, Cambridge, Mass.) and 5 μg of anti-LXRβ mouse antibody (Abcam, Cambridge, Mass.), while the remaining one quarter of the cross-linked chromatin from each sample was incubated with 5 μg of normal Rabbit IgG to serve as negative control.

The cross-linked chromatin samples were incubated overnight at 4° C. with their respective antibodies. The DNA-protein complexes were collected with Protein G agarose beads and washed to remove non-specific antibody binding. The DNA from the DNA-protein complexes from all the samples including the input and negative control was reverse cross-linked by incubation with 2 μL of Proteinase K for 2 hours at 65° C.

The crude DNA extract was eluted and then washed several times with wash buffer containing ethanol (provided with the kit) followed by purification with the use of DNA spin columns provided by the manufacturer. The pure DNA was eluted out of the DNA spin columns using 50 μL of the DNA elution buffer provided in the kit. 1 μL of the purified DNA was used for DNA concentration analysis using the “Quant-iT™ dsDNA Assay kit” from Invitrogen (Carlsbad, Calif.). The DNA fragment size was determined by electrophoresis on a 1.2% agarose FlashGel^{R} system (Lonza, Rockland, Me.).

The relative abundance of the ERα or ERβ antibody precipitated chromatin containing the ERE in the TH promoter region and LXRα or LXRβ antibody precipitated chromatin containing the LXRE in the α-synuclein promoter region was determined by qPCR using an iQ SYBR Green Supermix kit following the manufacturer's instructions (BioRad, Hercules, Calif.) and sequence specific primers (See Table 1 above). The amplification was performed using an iCycler iQ Multicolor Real Time PCR Detection System (BioRad, Hercules, Calif.). The fold enrichment of the ERE in the TH promoter region and LXRE in the α-synuclein promoter region was calculated using the ΔΔCₜ method (K. J. Livak et al., Methods, 25, 402 (2001)) which normalizes ChIP Cₜ values of each sample to the % input and background.

### H. Statistical Analysis

The significance of differences among the samples was assessed by One Way Analysis of Variance (One Way ANOVA) followed by Tukey's post-hoc test. Statistical analysis was performed with GraphPad Prism software 4.01. Quantitative data for Western blotting analysis are presented as mean values±S.E.M with unit value assigned to control and the magnitude of differences among the samples being expressed relative to the unit value of control. Quantitative data for RT-PCR analysis are presented as mean values±S.E.M, with reported values being the product of absolute value of the ratio of TH and α-synuclein mRNA to GAPDH mRNA multiplied by 1000000.

### Example 1

### TH Expression of THC is Reduced by 27-OHC and Estradiol Reverses the Effects of 27-OHC and Increases Basal Expression Levels of Tyrosine Hydroxylase

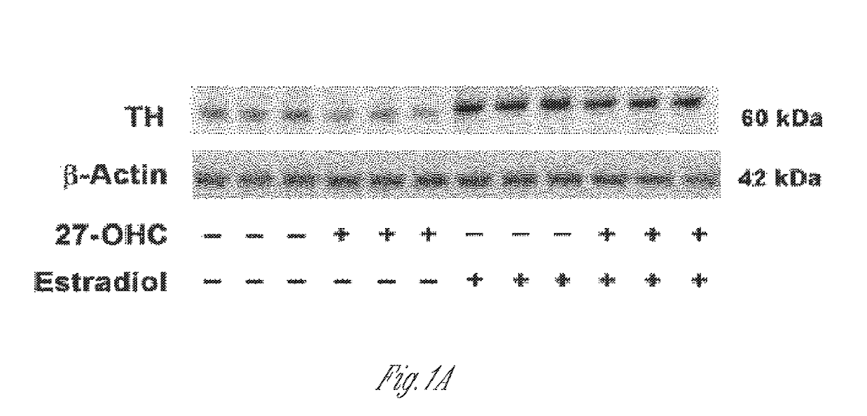

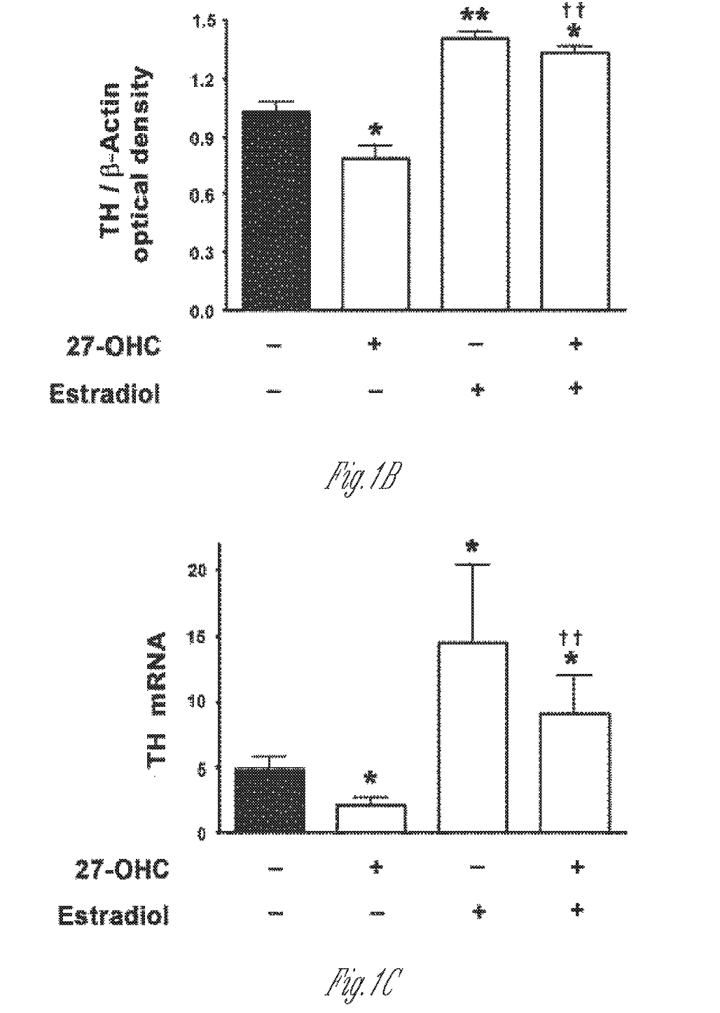

In this study, the involvement of ERα/β signaling in TH expression was evaluated and the effects of concomitant estradiol treatment on the 27-OHC-induced attenuation of TH expression levels were tested. Western blotting indicated that 27-OHC elicits a significant decrease in TH protein levels (FIG. 1a,b). Estradiol treatment resulted in a profound increase in TH protein levels.

Furthermore, concomitant treatment of SH-SY5Y neuroblastoma cells with 27-OHC and estradiol resulted in a significant increase in TH protein levels compared to control or 27-OHC treated samples (FIG. 1a,b). The complete reversal of 27-OHC-induced attenuation of TH expression by estradiol suggests that 27-OHC and estradiol may utilize a common downstream effector to modulate TH expression.

Subsequently it was whether or not these effects of 27-OHC and estradiol on TH expression were transcriptional in nature was investigated. Real Time RT-PCR analysis demonstrates that 27-OHC reduces the TH mRNA and this transcriptional attenuation is completely reversed by concomitant estradiol treatment (FIG. 1c). Furthermore, estradiol treatment elicits an increase in basal mRNA expression of TH (FIG. 1c).

### Example 2

### 27-OHC Reduces the Nuclear Translocation of ERα and ERβ and Estradiol Reverses the Effects of 27-OHC and Increases Basal Levels of ERα and ERβ in the Nucleus

Estrogens elicit their majority of effects by activating the cognate nuclear receptors ERα and ERβ. Estradiol is the most abundant and potent endogenous estrogen and activates both ERα and ERβ. Estradiol binding to the ER results in a conformational change that allows receptor dimerization (D. J. Mangelsdorf et al., Cell, 83, 835 (1995); M. Beato et al., Cell, 83, 851 (1995)). ER dimerization results in nuclear translocation, binding to the response element in the DNA, recruitment of co-activators or co-repressors and culminates into modulation of target gene expression. Id. 27-OHC has been characterized as a selective estrogen receptor modulator (SERM) (M. Umetani et al., Nat. Med., 13, 1185 (2007); C.D. DuSell et al., Mol. Endocrinol., 22, 65 (2008)). The potential of 27-OHC to modulate ER activity and subsequently regulate ER mediated TH expression was investigated.

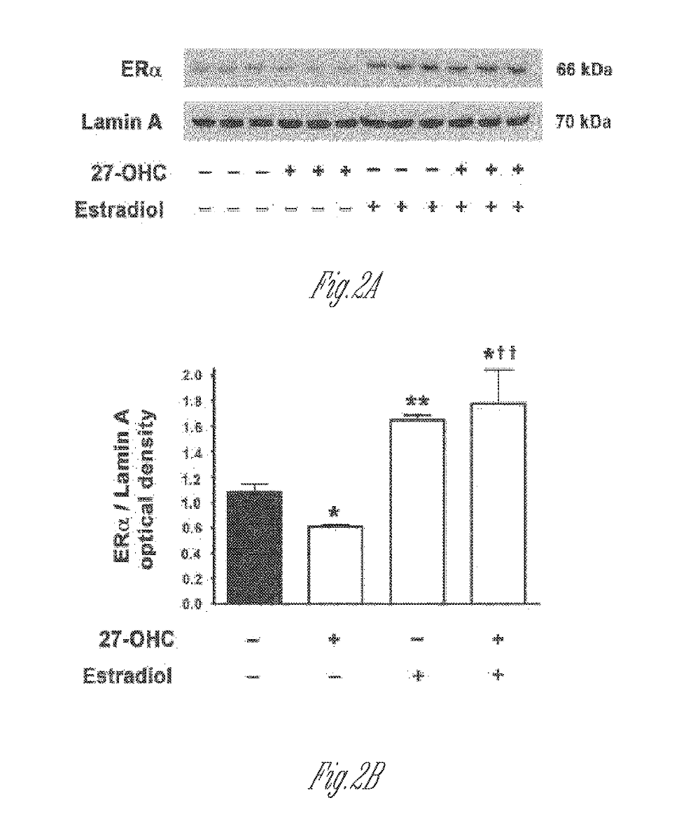

It was found that 27-OHC elicits a decrease in nuclear translocation of both ERα (FIG. 2a,b) and ERβ (FIG. 2c,d). These results suggest that 27-OHC possesses ER antagonist properties in the SH-SY5Y neuroblastoma cell line paradigm. Furthermore, treatment of cells with estradiol increased basal nuclear levels of ERα and completely reduced the 27-OHC-induced attenuation in the nuclear translocation of ERα (FIG. 2a,b) and ERβ (FIG. 2c,d).

### Example 3

### 27-OHC Reduces TH Expression by Attenuating the Binding of ER to ERE Half Site in the Promoter Region of TH. Treatment with Estradiol Reverses the Effects of 27-OHC

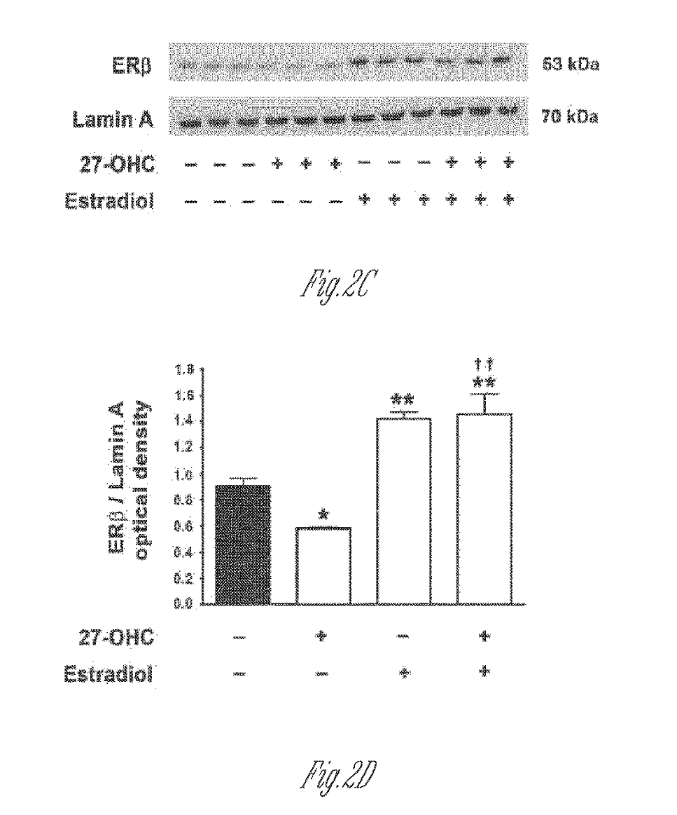

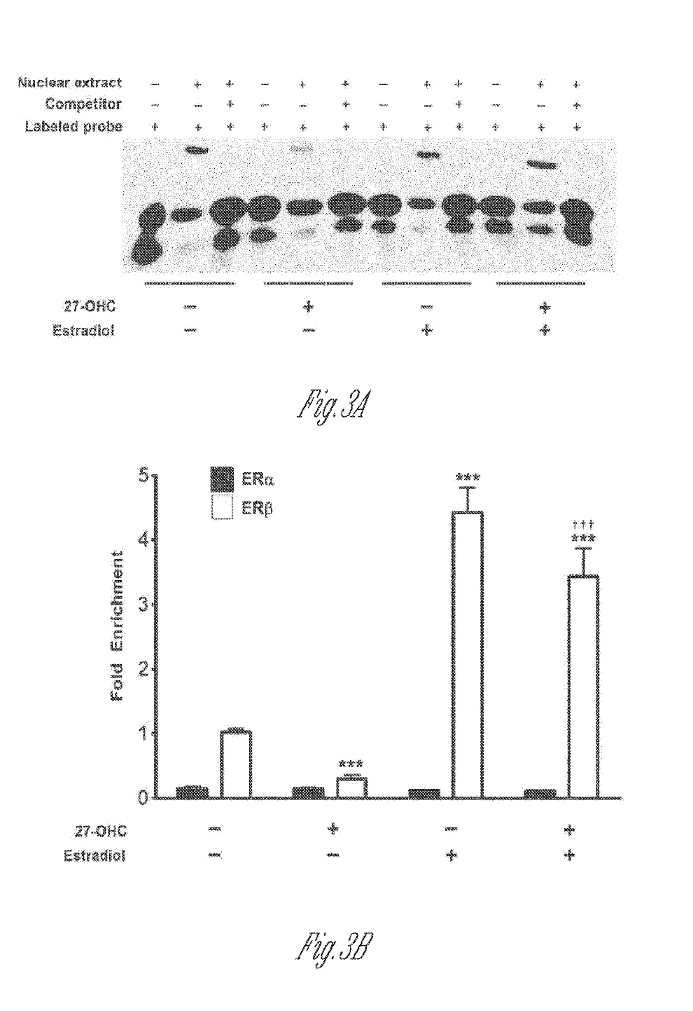

An attenuation was found in the nuclear translocation of ER in response to 27-OHC treatment, the binding of ER to the exogenous consensus sequence corresponding to the ERE half site in the TH promoter region was next investigated. To this end, Electrophoretic Mobility Shift Assay (EMSA) was performed using nuclear extracts from treated samples (FIG. 3a). A reduced binding of ER to the exogenous double stranded DNA probe representing the ERE half site on the TH promoter was found (FIG. 3a).

This could be a direct ramification of decreased ERα/β translocation to the nucleus, or decreased binding affinity of the translocated ERα/β to the ERE, or a combination of the two afore mentioned processes. Concomitant treatment with estradiol completely reverses the inhibitory effects of 27-OHC on binding of ER to the exogenous DNA probe (FIG. 3a).

The EMSA shows that there is “mobility shift” in each of the treated samples. However, the EMSA clearly depicts that the optical density of the shifted ER-DNA complex is of lower intensity in the 27-OHC treated cells compared to control. On the contrary, cells treated with a combination of 27-OHC and estradiol exhibit a profoundly greater intensity of the shifted ER-DNA complex. This clearly suggests that 27-OHC evokes a lower binding of nuclear ER to the exogenous double stranded DNA probe corresponding to the ERE-half site on the TH promoter.

Concomitant treatment with estradiol not only reverses, but profoundly increases binding of nuclear ER to the exogenous probe double stranded DNA probe corresponding to the ERE-half site on the TH promoter.

### Example 4

### 27-OHC-Induced Reduction in TH Expression is Mediated Via the Attenuation of ERβ Binding to ERE Half Site in the Promoter Region of TH and Subsequent Attenuation in ERβ Mediated TH Transcription

The EMSA analysis clearly revealed decreased binding of ER to the exogenous double stranded DNA probe corresponding to the ERE in the TH promoter. However, binding of ER to the ERE in the DNA is regulated by a host of epigenetic factors. EMSA does not account for other epigenetic modifications which occur concurrently and the presence of co-activators/co-repressors which regulate transcription. Furthermore, these epigenetic factors may regulate specific co-activators/co-repressors involved in TH expression and may also dictate the subtype of ER (ERα or ERβ) involved in the regulation of TH expression. To further characterize the effects of 27-OHC on the ERα/β binding to ERE on TH promoter and to elucidate the subtype of ER involved in the regulation of TH expression, we performed Chromatin Immunoprecipitaion (ChIP) analysis (FIG. 3b). Binding of ERβ to the ERE in the TH promoter was found to be about 10 times greater than binding of ERα to the ERE in TH promoter region in control samples (FIG. 3b). This suggests that in the basal state, the expression of TH is primarily responsive to ERβ.

Also 27-OHC treatment induced no significant attenuation of ERα binding to the ERE on the TH promoter (FIG. 3b), despite attenuating TH expression.

27-OHC treatment however did profoundly attenuate ERβ binding to the ERE on the TH promoter by 4-fold (FIG. 3b). Estradiol treatment, on the other hand, elicited a 4.5-fold increase in ERβ binding to the ERE half-site on the TH promoter, while producing no effect on ERα binding to the same site (FIG. 3b).

This suggests that the 27-OHC-induced attenuation in TH expression is primarily mediated via the inhibition of ERβ binding to the ERE in the TH promoter region.

Furthermore, treatment with estradiol concomitant to 27-OHC treatment increases ERβ, but not ERα, binding to the ERE on the TH promoter, corroborating the fact that ERβ regulates TH transcription and 27-OHC attenuates TH expression by reducing ERβ binding to TH promoter.

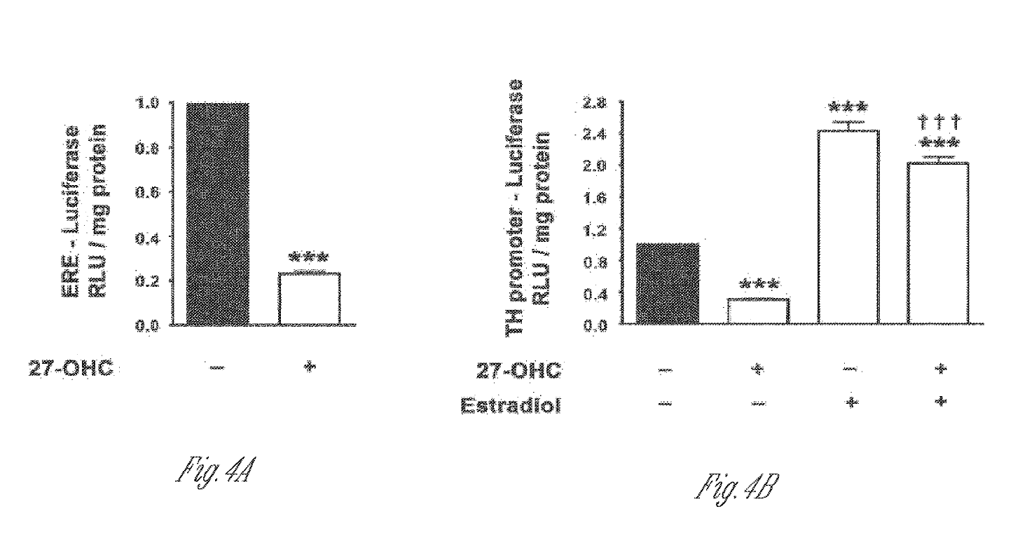

To further demonstrate that the changes in ERβ binding to the TH promoter indeed result in changes in transcription of TH, ER reporter assay and TH promoter analysis were done using a dual-luciferase assay system (FIG. 4a,b).

It was found that 27-OHC decreases ER-mediated transcription by ˜5-fold (FIG. 4a). Furthermore, 27-OHC reduced TH promoter activity by ˜4-fold and this effect was completely reversed by estradiol (FIG. 4b). Estradiol treatment also evoked a 2.5-fold increase in basal TH promoter activity (FIG. 4b).

### Example 5

### 27-OHC and the LXR Agonist GW3965 Increase α-Synuclein Expression and Treatment with the LXR Antagonist ECHS Reverses the 27-OHC-Induced Increase in α-Synuclein Expression

27-OHC is an endogenous ligand and activator of Liver X Receptors (LXR) α and β (B. A. Janowski et al., Nature, 383, 728 (1996)). There is evidence that α-synuclein expression is regulated by LXR signaling pathway and LXR consensus motifs, namely LXRE, are present in the α-synuclein promoter region (D. Cheng et al., Neuroreport, 19, 1685 (2008)).

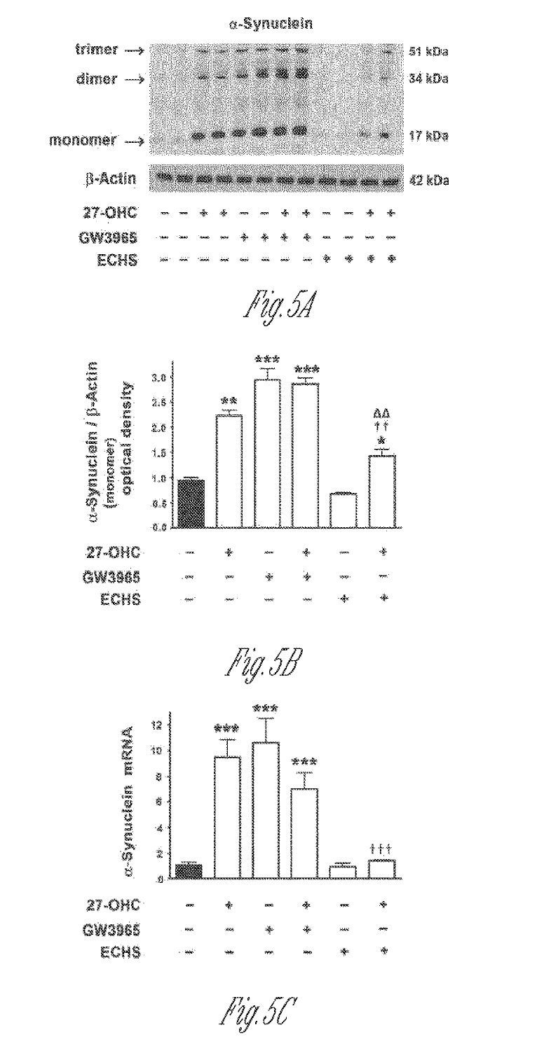

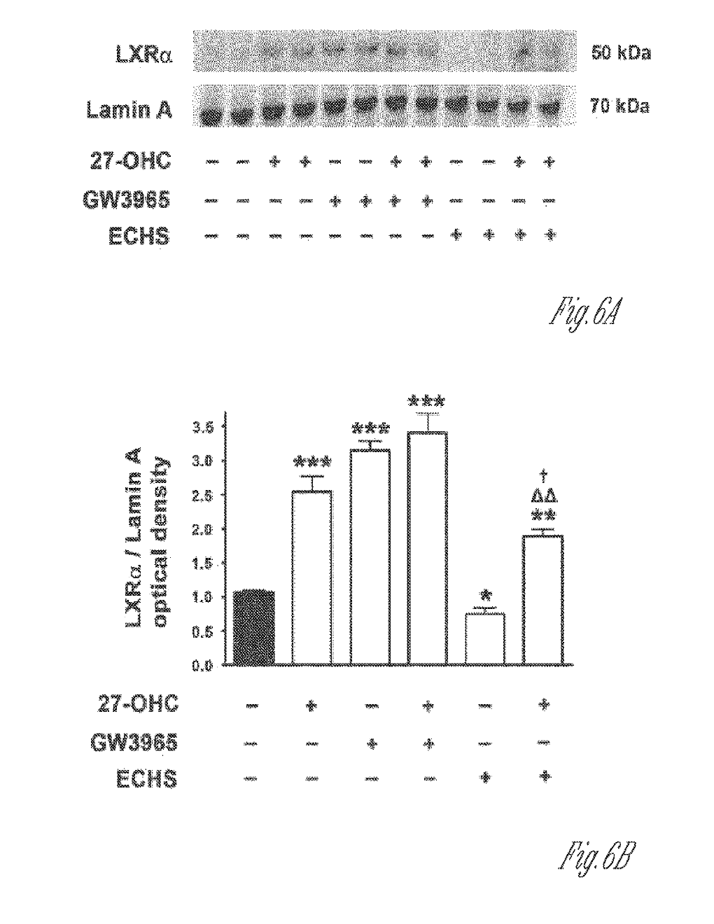

The effects of 27-OHC, LXR agonist GW3965, and a LXR antagonist ECHS on α-synuclein expression were determined. It was found that 27-OHC elicits a 2.3 fold increase in α-synuclein protein levels (FIG. 5a,b).

To investigate the involvement of LXR signaling in α-synuclein expression, the effects of a well characterized LXR agonist GW3965 (J. L. Collins et al., J. Med. Chem., 45, 1963 (2002); S. B. Joseph et al., PNAS USA, 99, 7604 (2002); N. Mitro et al., Nature, 445, 219 (2007)) and the LXR antagonist ECHS (C. Song et al., Steroids, 66, 473 (2001)) on α-synuclein expression were studied.

It was found that GW3965 increases α-synuclein expression levels by 3-fold, whereas the LXR antagonist ECHS does not produce any effect on basal α-synuclein expression levels (FIG. 5a,b). However, the LXR antagonist ECHS significantly attenuated the 27-OHC-induced increase in α-synuclein levels (FIG. 5a,b), thereby implicating LXR pathway in the 27-OHC-induced upregulation of α-synuclein expression.

To test the hypothesis that the effects of 27-OHC, GW3965, and ECHS are transcriptional in nature, a real time RT-PCR analysis was performed. It was found that an increase in α-synuclein mRNA expression with 27-OHC (9-fold) as well as GW3965 (10-fold) treatments and a marked attenuation in the 27-OHC-induced increase in α-synuclein mRNA with ECHS treatment (FIG. 5c). These results suggest that LXR pathway regulates the basal as well as the 27-OHC-induced increase in α-synuclein expression at the level of transcription.

### Example 6

### 27-OHC and GW3965 Increase LXRα/β Expression Levels in the Nucleus and ECHS Reverses the Effects of 27-OHC

LXRα and LXRβ are highly homologous transcription factors that belong to nuclear receptors family. 27-OHC, and other oxysterols such as 24,25-epoxycholesterol, are potent endogenous ligands and activators of LXRs (B. J. Janowski et al., cited above; J. M. Lehmann et al., J. Biol. Chem., 272, 3137 (1997)).

Ligand activated LXRs (LXRα and LXRβ) form heterodimers with retinoid X receptors (RXR) and modulate the expression of target genes by binding to the LXRE in the promoters of target genes. Furthermore, LXRα regulates its own transcription thereby suggesting an autoregulatory loop (B. A. Laffitte et al., Mol. Cell Biol., 21, 7558 (2001); K. D. Whitney et al., J. Biol. Chem., 276, 43509 (2001)). Endogenous LXRα/β ligands such as 20(S) hydroxycholesterol, 22(R) hydroxycholesterol and synthetic LXRα/β ligands GW3965 and TO901317 have been demonstrated to increase LXRα expression levels. Id.

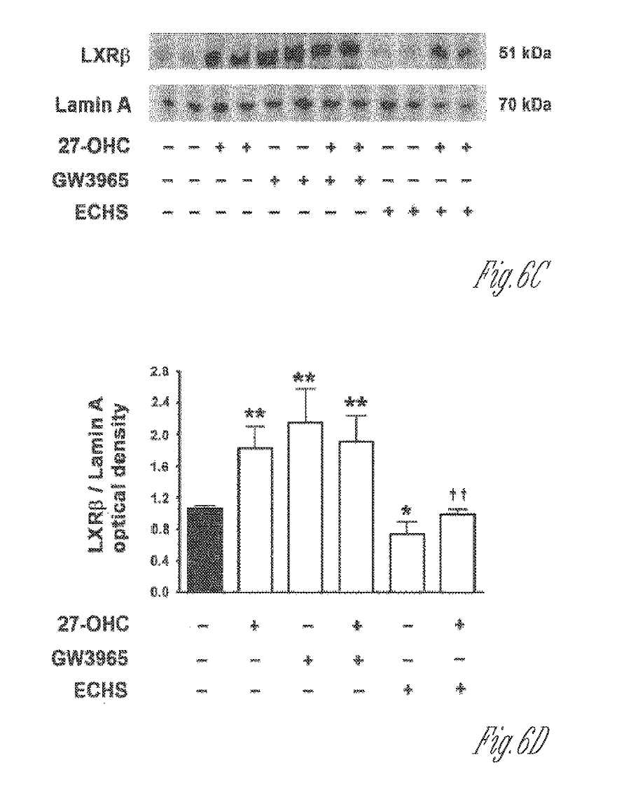

With 27-OHC characterized as an LXRα/β ligand and agonist, the effects of 27-OHC treatment on the expression levels of LXRα/β in the nucleus and the extent to which this may have an impact on LXR binding to the α-synuclein promoter were next investigated. LXRα/β expression levels in the nucleus in response to the synthetic agonist GW3965 and the antagonist ECHS were also determined. It was found that 27-OHC increases both LXRα (FIG. 6a,b) and LXRβ (FIG. 6c,d) protein levels in the nucleus. The synthetic LXRα/β agonist GW3965 exerted an analogous, but more pronounced increase in LXRα and LXRβ levels in the nucleus (FIG. 6a-d). On the other hand, treatment with LXRα/β antagonist ECHS reduced levels of LXRα and LXRβ in the nucleus (FIG. 6a-d). Furthermore, ECHS significantly attenuated the 27-OHC-induced increase in LXRα and LXRβ in the nucleus (FIG. 6a-d).

### Example 7

### 27-OHC and GW3965 Increase α-Synuclein Expression by Increasing the Binding of LXRα/β to LXRE in the Promoter Region of α-Synuclein, Effects Reversed by ECHS

To correlate the increased or decreased nuclear levels of LXRα/β with their transcriptional effects on target genes such as α-synuclein, an EMSA was performed to determine the extent to which treatments with 27-OHC, GW3965, or ECHS modulate LXRα/β binding to the exogenous double stranded DNA sequence, corresponding to the LXRE consensus motif on the α-synuclein promoter.

Two functional LXRE-SITE1 located 13788 nucleotides upstream (−13788) and the other SITE2 located 67043 nucleotides downstream (+67043) of the transcription start site have been identified in the α-synuclein promoter region (D. Cheng et al., cited above). A biotin labeled oligonucleotide probe corresponding to the LXRE located 13788 nucleotides upstream (−13788) of the transcription start site for the EMSA analysis was used.

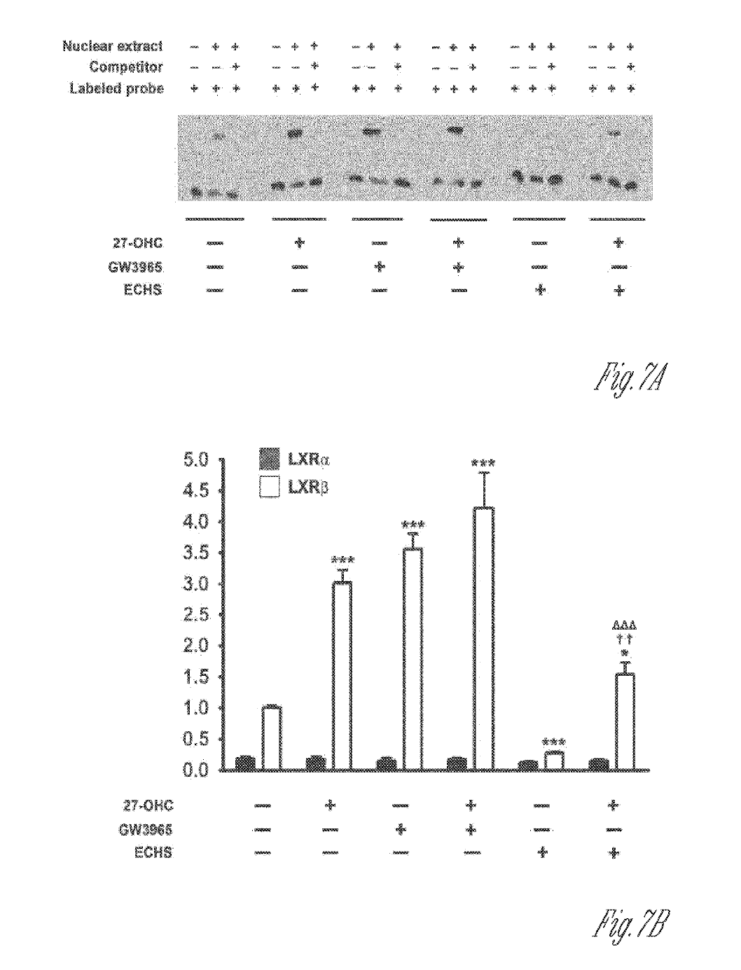

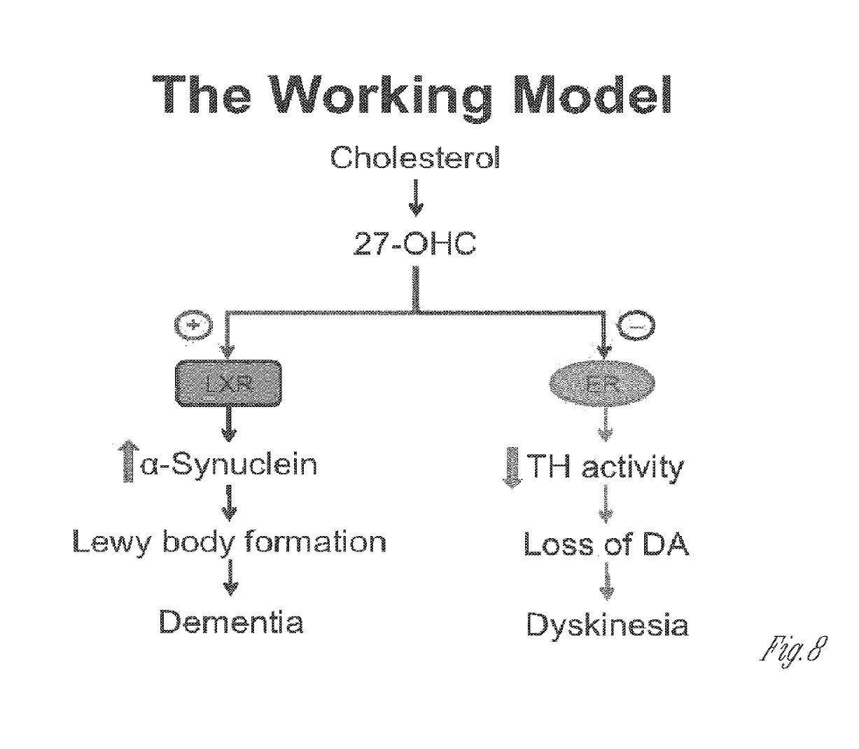

It was found that 27-OHC increases the binding of LXRα/β to the exogenous DNA sequence containing the LXRE (FIG. 7a) as demonstrated by an increase in optical density of LXRα/β-LXRE shifted complex, thereby positively correlating the increased LXRα/β nuclear levels with increased binding of LXRα/β to the exogenous DNA sequence comprising the LXRE consensus motif in the α-synuclein promoter.

Treatment with the synthetic LXRα/β agonist GW3965 exerted a similar increase in binding of LXRα/β to the exogenous DNA sequence containing the LXRE in the α-synuclein promoter (FIG. 7a). Treatment with the LXRα/β antagonist ECHS decreased binding of LXRα/β to the exogenous DNA sequence containing the LXRE (FIG. 7a). Furthermore, ECHS mitigates the 27-OHC-induced increase in LXRα/β binding to the LXRE consensus motif in the α-synuclein promoter (FIG. 7a).

### Example 8

### 27-OHC and GW3965 Increase α-Synuclein Expression by Increasing the Binding of LXRβ to LXRE in the α-Synuclein Promoter Region. ECHS Reverses the Effects of 27-OHC and GW3965

EMSA clearly depicts increased binding of LXRα/β to the exogenous DNA sequences representing the two LXRE consensus motifs in the α-synuclein promoter. However, LXRα/β mediated transcription is contingent on concomitant epigenetic modifications and the presence of co-activators/co-repressors that modulate LXRα/β binding to the LXRE on target genes.

Therefore, a ChIP assay was performed to determine the effects of 27-OHC, GW3965, and ECHS on LXRα/β binding to SITE1 LXRE in the α-synuclein promoter. ChIP analysis not only concurred and corroborated EMSA analysis data but also unveiled the LXR subtype involved in the α-synuclein promoter regulation.

The binding of LXRβ to the LXRE in the α-synuclein promoter is about 5 times higher than the binding of LXRα (FIG. 7b). This suggests that in the basal state, the expression of α-synuclein is primarily responsive to LXRβ relative to LXRα. Furthermore, 27-OHC treatment induced no significant augmentation of LXRα binding to the LXRE on the α-synuclein promoter (FIG. 7b) despite augmenting α-synuclein expression.

27-OHC treatment however did profoundly elevate LXRβ binding to the LXRE (˜3 fold) in the α-synuclein promoter (FIG. 7b).

This suggests that the 27-OHC-induced increase in α-synuclein expression is primarily mediated via the activation of LXRβ binding to the LXRE in the α-synuclein promoter region. Treatment with the LXR agonist GW3965, either alone or in combination with 27-OHC, increases LXRβ binding to the LXRE on the α-synuclein promoter by 3.5-fold and 4-fold respectively (FIG. 7b). However, the LXR agonist GW3965, either alone or in combination with 27-OHC, did not elicit any increase in LXRα binding to the LXRE in the α-synuclein promoter (FIG. 7b). This further implicates LXRβ in the regulation of α-synuclein expression.

Furthermore, treatment with the LXR antagonist ECHS resulted in a ˜2.8 fold decrease in LXRβ binding to the LXRE on the α-synuclein promoter (FIG. 7b). However, ECHS did not significantly attenuate LXRα binding to the LXRE on the α-synuclein promoter (FIG. 7b). This further corroborates the fact that LXRβ mediates basal as well as 27-OHC-induced increase in α-synuclein expression. Furthermore, ECHS significantly reversed the 27-OHC-induced increase in LXRβ binding to the LXRE in the α-synuclein promoter (FIG. 7b).

### Example 9

### LXR Signaling Pathway does not Regulate TH Expression in Human SH-SY5Y Neuroblastoma Cells

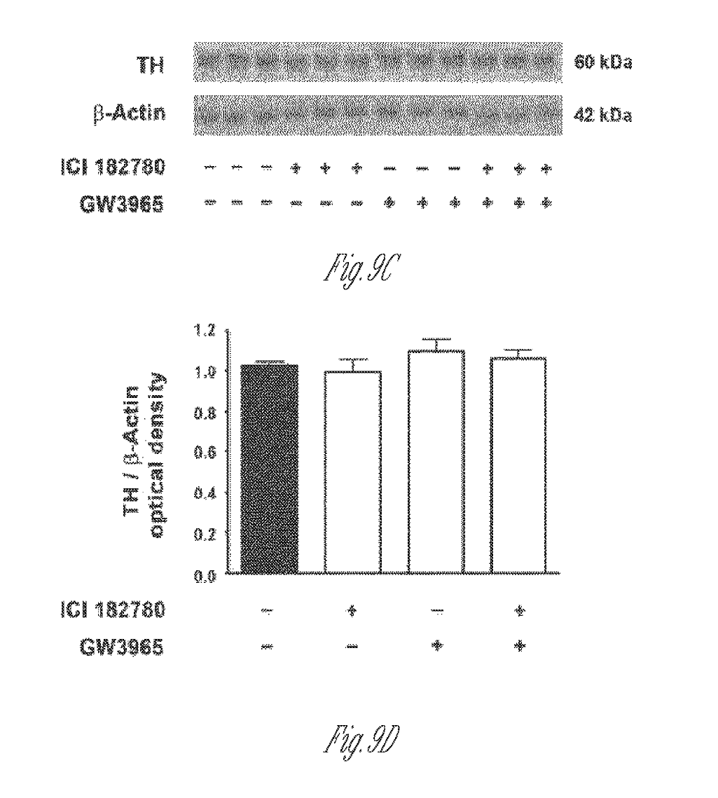

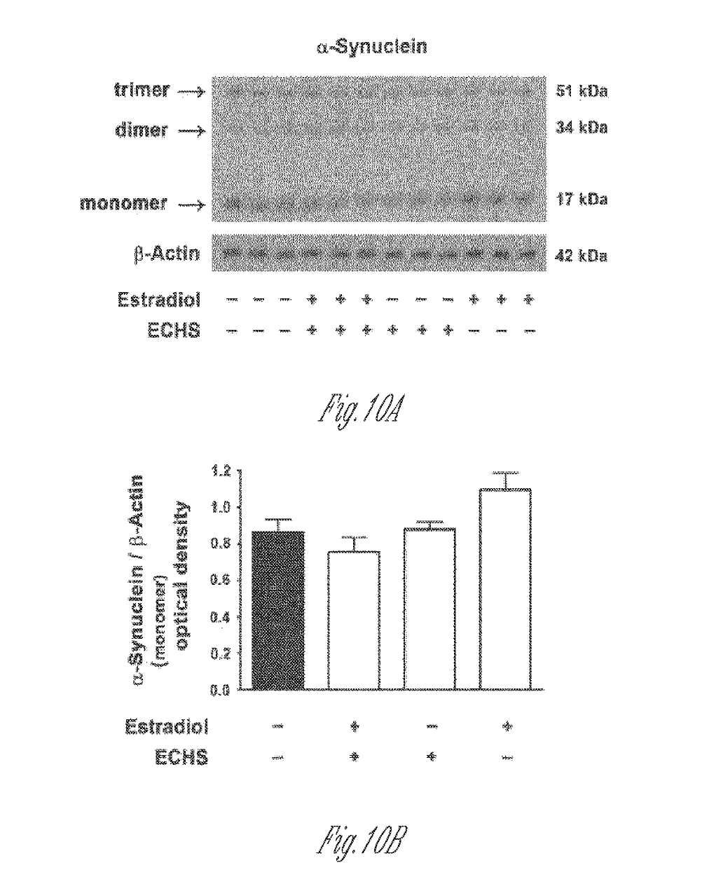

FIG. 9 depicts the effects of a combination of estradiol (ER agonist) and ECHS (LXR antagonist) on TH expression levels in human SH-SY5Y neuroblastoma cells. In FIG. 9, Western blotting panel (a) and densitometric analysis, panel (b), clearly demonstrate that the LXR antagonist ECHS either alone or in combination with estradiol has no effect on TH expression. Panels (c,d) depict the effects of combination of ICI 182780 (ER antagonist) and GW3965 (LXR agonist) on TH expression levels in human SH-SY5Y neuroblastoma cells. Western blotting panel (c), and densitometric analysis, panel (d), demonstrate that the LXR agonist GW3965 elicits no effect on TH expression.

### Example 10

### ER Signaling Pathway does not Regulate α-Synuclein Expression in Human SH-SY5Y Neuroblastoma Cells

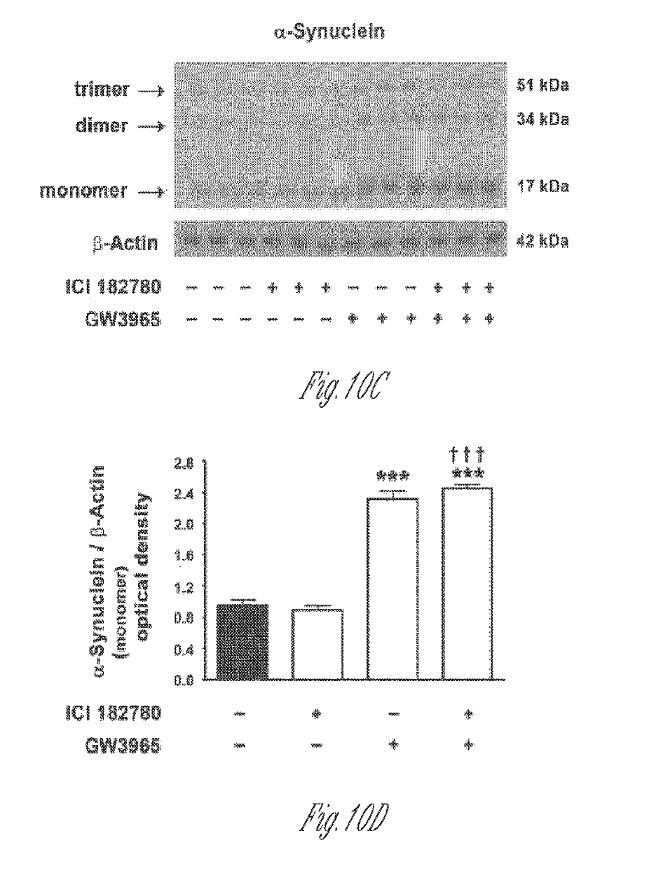

FIG. 10 (a,b) depicts the effect of a combination of estradiol (ER agonist) and ECHS (LXR antagonist) on α-synuclein expression levels in human SH-SY5Y neuroblastoma cells. Western blotting, panel (a), and densitometric analysis, panel (b), clearly demonstrate that the ER agonist estradiol either alone or in combination with ECHS has no effect on α-synuclein expression.

FIG. 10 (c,d) depicts the effects of a combination of ICI 182780 (ER antagonist) and GW3965 (LXR agonist) on α-synuclein expression levels in human SH-SY5Y neuroblastoma cells. Western blotting, panel (c), and densitometric analysis, panel (d) demonstrate that the ER antagonist ICI 182780 elicits no effect on α-synuclein expression.

### Example 11

### LXR Antagonist ECHS does not Affect the Estradiol Mediated Mitigation of 27-OHC-Induced Attenuation in TH Expression, while Estradiol does not Affect the ECHS Mediated Attenuation of 27-OHC-Induced Increase in α-Synuclein Expression

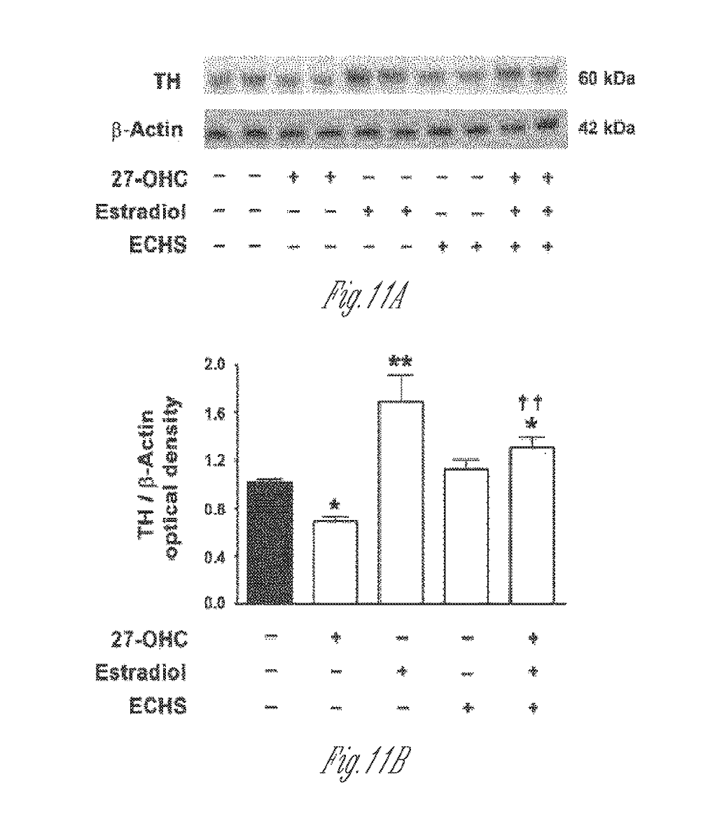

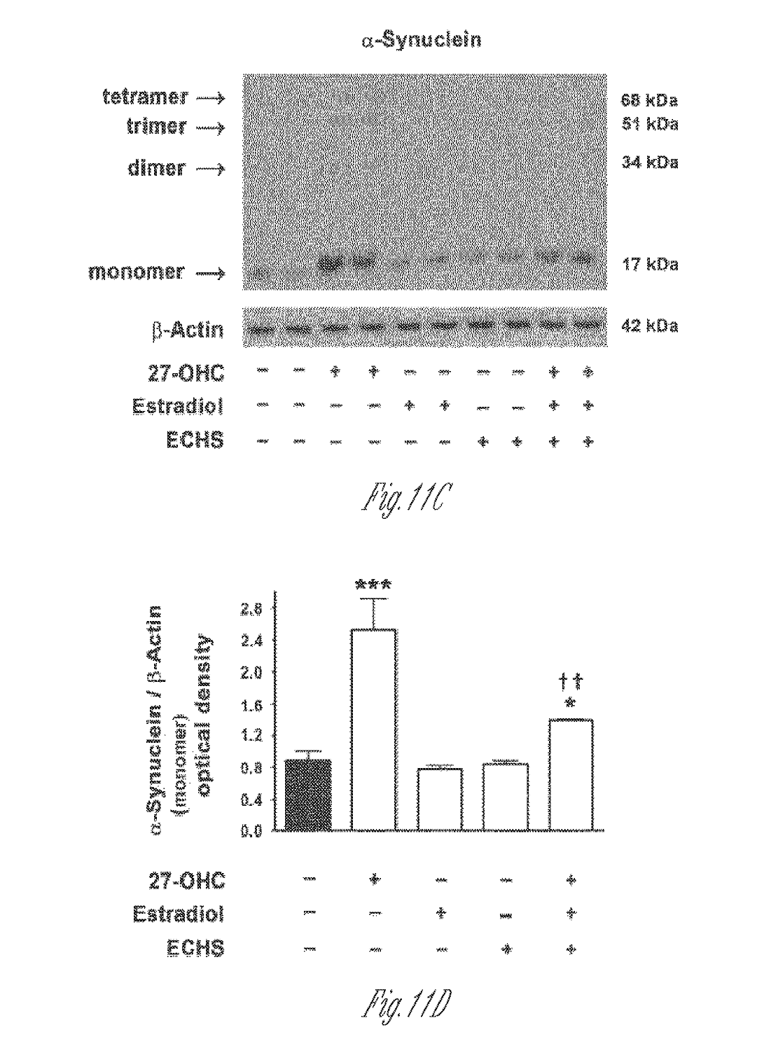

FIG. 11 depicts the effects of a combination of estradiol (ER agonist), ECHS (LXR antagonist) and 27-OHC on TH and α-synuclein expression levels in human SH-SY5Y neuroblastoma cells. Western Blotting (a) and densitometric analysis (b) clearly demonstrates that the LXR antagonist ECHS does not alter the effect of estradiol on 27-OHC-induced decrease in TH expression (compare with FIGS. 1a and 1b). Both sets of treatments (27-OHC-+Estradiol and 27-OHC+Estradiol+ECHS) depict a ˜40% increase in TH relative to basal levels.

Western blotting (c) and densitometric analysis (d) clearly demonstrate that estradiol does not alter the effect of ECHS on 27-OHC-induced increase in α-synuclein expression (compare with FIGS. 5a and 5b). Both sets of treatments (27-OHC+ECHS and 27-OHC+Estradiol+ECHS) yield ˜45% higher α-synuclein levels than basal expression levels.

### Prophatic Example 12

### Determine the Extent to which HO-1 Inhibitors, ER Agonists and LXR Antagonists Protect Against PD-Like Pathology

### (a) Introduction

Our preliminary data shows that the oxysterol 27-OHC reduces TH, increases α-synuclein, and causes cell death (Rantham Prabhara et al., cited above). It has also been demonstrated that oxysterols are elevated in the cerebral cortex of individuals with Lewy body dementia relative to those of age-matched controls (Bosco et al., cited above). This suggests that oxysterols may accelerate α-synuclein aggregation.

In contrast to 24-OHC, which is almost exclusively produced in the brain and crosses to the circulation as a route of elimination of excess cholesterol, 27-OHC is produced in most organs, including the brain, but is the most abundant cholesterol metabolism in the circulation.

It is expected that increased entry into the brain, overproduction in the brain or reduced clearance from the brain of 27-OHC causes the accumulation of this oxysterol in this organ. Evidence of increased 27-OHC or reduced 24-OHC:27-OHC ratio in substantia nigra of PD patients is yet to be demonstrated. Heme-oxygenase 1 (HO-1) has been shown to enhance the conversion of cholesterol to oxysterols, and it is expected that 27-OHC levels can be increased in the presence of high levels of HO-1. It has previously been shown that hypercholesterolemia increases oxidative stress and HO-1 levels. It is possible that increase oxidative stress and subsequent increases in HO-1 levels following hypercholesterolemia facilitates the conversion of cholesterol to 27-OHC.

Plasma levels of 27-OHC increase from 70 ng/ml in controls to 340 ng/ml in cholesterol-fed rabbits (n=6). Increased levels of 27-OHC would inhibit ERs and over-activate LXRs, thus increasing α-synuclein production, reducing TH levels and potentially contributing to the pathogenesis of PD. It is expected that completion of this example will demonstrate the extent of the deleterious effects of cholesterol dyshomeostasis and the beneficial protective effects of HO-1 inhibitors, ER agonists and LXR antagonists in PD models.

### (b) Preliminary Data.

Cholesterol-enriched diets increase α-synuclein and reduce TH immunoreactivity in rabbit substantia nigra. Rabbits were fed a 2% cholesterol-enriched diet for 12 weeks and were examined to see the extent to which this diet affects 27-OHC, 24-OHC, α-synuclein and TH levels in substantia nigra of these animals. Results: (i) Both 24-OHC and 27-OHC levels are markedly increased in the plasma. In the brain, the 27-OHC/24-OHC ratio increased from 1:100 to 1:150. As the concentrations of these oxysterols are tightly regulated in the brain, the increase in the 27-OHC/24-OHC ratio may have severe effects on the brain. (ii) Immunoreactivity for α-synuclein increases and TH staining substantially decreased in substantia nigra from cholesterol-fed rabbits.

These results show that long-term dietary intake of cholesterol-enriched food causes pathological hallmarks of relevance to PD.

### (c) Experimental Approach.

This example will first determine the extent to which a high-fat diet increases HO-1 levels, increases 27-OHC levels, and induces PD-like pathology in wild type mice. Next, the extent to which a high-fat diet exacerbates PD-like pathology by increasing HO-1 levels, increasing 27-OHC levels, reducing TH expression levels and accelerating α-synuclein accumulation in the α-synuclein transgenic mice model for familial PD that does not exhibit TH reduction under normal diet will be determined Third, the extent to which inhibiting HO-1 will preclude 27-OHC increase, TH reduction and α-synuclein accumulation in wild type and in α-synuclein transgenic mice will be determined Fourth, the potential protective effects of ER agonists and LXR antagonists in MPTP-treated mice model that exhibits TH reduction, α-synuclein accumulation and PD-like symptoms will be evaluated.

(i) does HO-1 Inhibition Protect Against PD-Like Pathology in Animals Fed a High-Fat Diet?

In this study, it will be determined if fat intake induces PD in wild type mice and exacerbates genetic susceptibility to PD in α-synuclein transgenic mice. The effect of the HO-1 inhibitor Tin-mesoporphyrin IX (SnMP) on PD-like pathology in these mice will be measured.

(ii) Mice:

Adult male mice will be assigned to 8 groups (10 months old; n=10 each group). Group 1: Control (B6C3F1/J) mice fed normal chow. Group 2: B6C3F1/J mice fed normal chow and treated with OH-inhibitor Stannous mesoporphyrin (SnMP). Group 3: B6C3F1/J fed a high-fat diet. Group 4: B6C3F1/J fed a high-fat diet and treated with SnMP. Group 5: B6; C3-Tg(Prnp-SNCA*A53T)83Vle/J fed normal chow. Group 6: B6; C3-Tg(Prnp-SNCA*A53T)83Vle/J fed normal chow and treated with SnMP. Group 7: B6; C3-Tg(Prnp-SNCA*A53T)83Vle/J fed a high-fat diet. Group 8: B6; C3-Tg(Prnp-SNCA*A53T)83Vle/J fed a high-fat diet and treated with SnMP.

(iii) Treatments:

The high-fat diet (35% fat from lard, 58.4% kcal from fat; Harlan Teklad, Madison, Wis.) will be initiated at 10 months of age and continued for 12 weeks, at which time mice are euthanized. SnMP (Protoporphyrin products; Logan, Utah) is dissolved in 0.5 ml 0.5 M NaOH and then 3 ml of lactated Ringer's solution is added and the mixture is adjusted to a pH of 7.8 by addition of 0.1M HCl. The solution is prepared freshly every day, protected from light and filtered before administration through sterile Nylon membrane of 0.2 μm pore size. SnMP will be administered at 10 μmol.Kg⁻¹.day⁻¹ i.p. for 4 weeks starting 8 weeks after the initiation of the high-fat diet. SnMP crosses the blood brain bather and has been shown to protect against experimental intracerebral hemorrhage at 10 μmol.Kg⁻¹.day⁻¹ i.p.

### (d) do an LXR Antagonist and an ER Agonist Protect Against PD-Like Pathology in MPTP Mouse Model?

This study will determine the extent to which the ER agonist 17β-estradiol (E2; Sigma) and the LXR antagonist ECHS (5α,6α,-epoxycholesterol-3-sulfate; Steraloids, Inc., Newport, R.I.) protect against PD-like pathology in MPTP-treated mice. This MPTP model shows structural, morphological and functional features relevant to human PD.

(i) Mice:

Adult male C57BL/6 mice (12 months old; 25-30 g) will be fed normal chow and assigned to 4 groups (n=10 each). Group 1: MPTP-treated mice; Group 2: MPTP-treated mice plus the LXR antagonist ECHS; Group 3: MPTP-treated mice plus the ER agonist E2; and Group 4: MPTP-treated mice plus (ECHS+E2).

(ii) Treatments:

MPTP-HCl (Sigma; St. Louis, Mo.) will be injected i.p. in the mice at a dose of 18 mg/kg (freshly dissolved in sterile 0.9% saline) twice the first 2 days at an interval of 12 h and then once a day for three more consecutive days. This protocol has been shown to affect all aspects of the dopamine system within the substantia nigra. The LXR antagonist ECHS and the ER agonist E2 will be infused at a dose of 0.1 or 1 μg.kg⁻¹.day⁻¹, either individually or combined, via a cannula stereotaxically implanted into the dorsal third ventricle of mice one day after the last injection of MPTP and connected to an Alzet osmotic pump (Cupertino, Calif.; model 1002; delivery rate of 0.25 μl/h). Treatments will be infused for 14 days. Control MPTP-injected mice that do not receive ECHS and/or E2 are infused with the same volume of saline as treated mice.

(iii) Methodology and Data Analyses:

Mice will undergo behavioral assessment after the last injection of MPTP and at the end of treatment with ECHS and/or E2 before being killed. At necropsy, mice will be perfused with Dulbecco's phosphate-buffered saline and the brains will be promptly removed and cut to yield two symmetrical hemispheres; one will be processed immediately for Western blot and real time RT-PCR analyses and the other half is snap frozen for immunohistochemistry.
(1) Behavioral assessment: Mice from Study A and Study B will undergo behavioral assessment using the open field test, a common method to evaluate locomotion and rearing which are indicative of hypodyskenesia in MPTP-treated mice.
(2) Western blot analyses: Substantia nigra dissected from each mouse will be processed with similar techniques as described in Aim 2. The following antibodies will be used: anti-TH (1:100), anti-phospho Ser⁴⁰-TH rabbit polyclonal antibody (1:1000), anti-α-synuclein rabbit monoclonal antibody (1:100), and anti-HO-1 antibody (1:100). Additionally, antibody to ABCA1 (1:100) which is an LXR target protein and to cyclin D1 (1:100) an ER target protein will be used.
(3) Real Time RT-PCR: Total RNA is isolated from substantia nigra using 5 prime “PerfectPure RNA tissue kit” (5 Prime, Inc.). cDNA is obtained by reverse transcribing 1 μg of extracted RNA using an iScript cDNA synthesis kit. Primers will be used to amplify the TH and α-synuclein mRNA. The cDNA amplification will be performed using an iQ SYBR Green Supermix kit. The expression of specific TH and α-synuclein transcripts amplified is normalized to the expression of glyceraldehyde-3-phosphate dehydrogenase (GAPDH).
(4) For immunohistochemistry of TH and α-synuclein: Frozen coronal sections (10 μm thick) cut at the level of substantia nigra from mice are fixed in 4% paraformaldehyde, washed in PBS, blocked with normal goat serum and incubated overnight with anti-TH (1:500), anti-phospho TH (1:500) or α-synuclein antibodies (1:500). Sections are incubated with biotinylated secondary antibody followed by HRP streptavidin, and visualized by DAB. All sections will be visualized under a light microscope. TH- and α-synuclein-positive staining will be double-blind counted in the substantia nigra.
(5) Cholesterol and oxysterol measurements: Total plasma cholesterol, HDL, LDL, and triglycerides will be measured. Concentrations of cholesterol, 24-OHC and 27-OHC in substantia nigra using Mass Spectrometry will be determined
(6) Dopamine and dopamine metabolite measurements: Because changes in levels of TH affect dopamine and noradrenaline levels, the concentrations of dopamine and its metabolites DOPAC and HVA as well as noradrenaline will be determined.

(iv) Statistical Analyses.

All data will be expressed as mean±SEM. Statistically significant differences between the groups will be analyzed using ANOVA followed by Tukey's post-hoc test for comparison of multiple treatments. Statistical analysis will be performed with GraphPad Prism software 4.01. A probability value of ≦0.05 is considered as significant.

### (e) Expected Outcomes.

The preliminary data shows that fat intake increases α-synuclein and reduces TH levels in rabbit substantia nigra. The extent to which fat intake will reproduce similar effects in wild type mice will be determined According to literature, this diet has been shown to affect expression of genes regulating the availability of dopamine in mice. Information will also be obtained on the extent to which high-fat diet exacerbates genetic susceptibility to PD in the α-synuclein transgenic mice by increasing locomotor dysfunction, reducing TH levels and fostering α-synuclein accumulation. This example is expected to demonstrate that the high-fat-food intake leads to increased HO-1, 27-OHC and α-synuclein, as well as reduce TH levels in substantia nigra of wild type and transgenic mice. The HO-1 inhibitor Tin-mesoprorphyrin IX (SnMP) is expected to reduce locomotor dysfunction and excess conversion of cholesterol to 27-OHC, thus precluding α-synuclein accumulation and TH level reduction.

Mice subjected to the subacute MPTP injections will show locomotor dysfunction as well as reduced TH and increased α-synuclein levels. Treatment of the MPTP-injected mice with the LXR antagonist ECHS is expected to reduce increased α-synuclein levels, whereas treatment with the ER agonist 17β-estradiol (E2) is expected to prevent TH reduction. Treatment with both ECHS and E2 will concomitantly reduce locomotor dysfunction and α-synuclein accumulation and prevents TH reduction. Such results will demonstrate that modulating both ERs and LXRs is required to regulate levels of both α-synuclein and TH. Successful completion of this example will provide information on factors and mechanisms by which these factors modulate the expression levels of TH and α-synuclein, two proteins that are central to the pathophysiology of PD. Agents that act on ERs and/or LXRs can be designed and screened to determine their protective effects in preclinical studies for PD.

### Example 13

### Cholesterol-Enriched Diet Causes Age-Related Macular Degeneration-Like Pathology in Rabbit Retina

### A. Background.

Alzheimer's disease (AD) and age-related macular degeneration (AMD) share several pathological hallmarks including β-amyloid (Aβ) accumulation, oxidative stress, and apoptotic cell death. The causes of AD and AMD are likely multi-factorial with several factors such as diet, environment, and genetic susceptibility participating in the pathogenesis of these diseases. Epidemiological studies correlated high plasma cholesterol levels with high incidence of AD, and feeding rabbits with a diet rich in cholesterol has been shown to induce AD-like pathology in rabbit brain. High intake of cholesterol and saturated fat were also long been suspected to increase the risk for AMD. However, the extent to which cholesterol-enriched diet may also cause AMD-like features in rabbit retinas is not well known.

### B. Methods.

Male New Zealand white rabbits were fed normal chow or a 2% cholesterol-enriched diet for 12 weeks. At necropsy, animals were perfused with Dulbecco's phosphate-buffered saline and the eyes were promptly removed. One eye of each animal was used for immunohistochemistry and retina dissected from the other eye was used for Western blot, ELISA assays, spectrophotometry and mass spectrometry analyses.

### C. Results.

Increased levels of Aβ, decreased levels of the anti-apoptotic protein Bcl-2, increased levels of the proapoptotic Bax and gadd153 proteins, emergence of TUNEL-positive cells, and increased generation of reactive oxygen species were found in retinas from cholesterol-fed compared to normal chow-fed rabbits. Additionally, astrogliosis, drusen-like debris and cholesterol accumulations in retinas from cholesterol-fed rabbits were observed. As several lines of evidence suggest that oxidized cholesterol metabolites (oxysterols) may be the link by which cholesterol contributes to the pathogenesis of AMD, the levels of oxysterols were determined and a dramatic increase was found in the levels of oxysterols in retinas from cholesterol-fed rabbits.

### D. Conclusions.

These results suggest that cholesterol-enriched diets cause retinal degeneration that is relevant to AMD. Furthermore, our data suggests high cholesterol levels and subsequent increase in the cholesterol metabolites as potential culprits in AMD. See B. Dasani, BMC Ophthalmology, 11, 20 (2011).

### Example 14

### The Oxysterol 27-Hydroxycholesterol Increases β-Amyloid and Oxidative Stress in Retinal Pigment Epithelia Cells

A. Background.

Alzheimer's disease (AD) and age-related macular degeneration (AMD) share several pathological features including β-amyloid (Aβ) peptide accumulation, oxidative damage, and cell death. The causes of AD and AMD are not known but several studies suggest disturbances in cholesterol metabolism as a causative factor in these diseases. It has recently been shown that the cholesterol oxidation metabolite 27-hydroxycholesterol (27-OHC) causes AD-like pathology in human neuroblastoma SH-SY5Y cells and in organotypic hippocampal slices. However, the extent to which and the mechanisms by which 27-OHC may also cause pathological hallmarks related to AMD are ill-defined. In this study, the effects of 27-OHC on AMD-related pathology were determined in ARPE-19 cells. These cells have structural and functional properties relevant to retinal pigmented epithelial cells, a target in the course of AMD.

### B. Methods.

ARPE-19 cells were treated with 0, 10 or 25 μM 27-OHC for 24 hours. Levels of Aβ peptide, mitochondrial and endoplasmic reticulum (ER) stress markers, Ca²⁺ homeostasis, glutathione depletion, reactive oxygen species (ROS) generation, inflammation and cell death were assessed using ELISA, Western blot, immunocytochemistry, and specific assays.

### C. Results.

27-OHC dose-dependently increased Aβ peptide production, increased levels of ER stress specific markers caspase 12 and gadd153 (also called CHOP), reduced mitochondrial membrane potential, triggered Ca²⁺ dyshomeostasis, increased levels of the nuclear factor κB (NFκB) and heme-oxygenase 1 (HO-1), two proteins activated by oxidative stress. Additionally, 27-OHC caused glutathione depletion, ROS generation, inflammation and apoptotic-mediated cell death.

### D. Conclusions.

The cholesterol metabolite 27-OHC is toxic to RPE cells. The deleterious effects of this oxysterol ranged from Aβ accumulation to oxidative cell damage. These results suggest that high levels of 27-OHC may represent a common pathogenic factor for both AMD and AD. See B. Dasani et al., BMC Ophthalmology, 10, 22 (2010).

### Prophetic Example 15

### Determine the Extent to which LXR Antagonists and ER Agonists Protect Against AMD Pathology in Mouse Model that Exhibits Morphological, Ultrastructural and Functional Features of AMD

### A. Rationale.

27-OHC is an LXR agonist and is also an ER antagonist. Activation of LXRs and inhibition of ERs would potentially contribute to the generation of AMD-like pathology. Both LXRs and ERs are expressed in retina. Inhibition of LXRs and activation of ERs would reverse the 27-OHC-induced deleterious effects. The goal of this example is to demonstrate the extent to which the synthetic LXR antagonist 5α,6α,-epoxy cholesterol-3-sulfate (ECHS) and the ER agonist 17β-estradiol (E2) prevent or reduce AMD pathology in mice models of AMD. It is believed that completion of this example will demonstrate the cellular mechanisms involved in the deleterious effects of 27-OHC and the beneficial effects of agents that can oppose 27-OHC by regulating these cellular pathways.

### B. Preliminary Data.

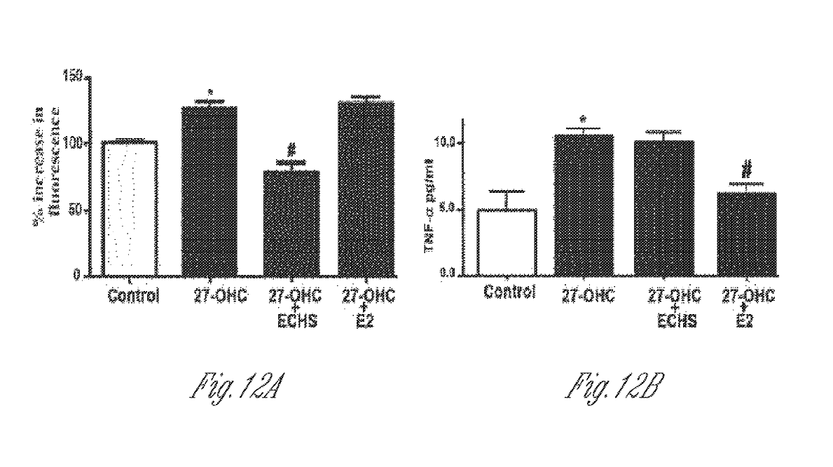

ARPE-19 cells were incubated with 27-OHC in the presence or absence of the LXR antagonist ECHS and the ER agonist E2. As shown in FIG. 12, co-treatment with ECHS reduced 27-OHC-induced increase in ROS and co-treatment with E2 reduced 27-OHC-induced increase in TNF-α levels. Treatment of ARPE-19 cells for 24 h with 27-OHC increases reactive oxygen species (ROS) (left panel) and TNF-α levels (right panel). Co-treatment with ECHS, but not E2, reduced ROS levels. Co-treatment with E2, but not ECHS, reduced the increased TNF-α levels. This preliminary data suggests that the deleterious effects of 27-OHC involve two distinct pathway, LXRs and ERs, and that protection against 27-OHC-induced effects requires activation of ERs and inhibition of LXRs.

### C. Experimental Approach.

Ccl-2^{-/-} mice (B6. 129S4-Ccl2^{tmlRol}/J; Stock Number: 004434) are purchased from Jackson Laboratory and bred and genotyped in the Center for Biomedical Research facility at the University of North Dakota School of Medicine for developing a colony. These mice develop a significant number of drusen by 12 months of age and exhibit morphological, ultrastructural and functional features characteristic of human AMD. The LXR antagonist ECHS and the ER agonist E2 will be administered either individually or combined and their effects on AMD-like pathology will be determined.

(1) Animals and Treatments.

Male Ccl-2^{-/-} mice will be administered the LXR antagonist ECHS (Steraloids, Inc., Newport, R.I.) at 10 μM and 100 μM and/or the ER agonist 17β-estadiol or E2 (Sigma) at 2 μg/day/mouse. ECHS and E2 will be dissolved in dimethyl sulfoxide (1% DMSO in saline) and administered i.p. in 0.2 ml volume either individually or in combination for 12 weeks starting at 9 months of age. Mice are randomly assigned to the following groups:

| | | | | | ECHS | ECHS |
|---|---|---|---|---|---|---|
| | | ECHS | | | (10 μM) + | (100 μM) + |
| | Vehicle | 10 μM | 100 μM | E2 | E2 | E2 |
| Ccl-2^{-/-} | (n = 10) | (n = 10) | (n = 10) | (n = | (n = 10) | (n = 10) |
| | | | | 10) | | |

Control non-treated mice will be injected with same volume of saline as treated mice.

(2) Methods and Data Analyses.

By the end of the treatment periods, animals will be perfused with Dulbecco's phosphate-buffered saline and eyes promptly enucleated and subjected to the following analysis.

(i) Structural changes in retinas Immunohistochemistry for light and fluorescent microscopy will include staining with an antibody to GSA, hematoxylin & eosin (H&E), vitronectin, lipofuscin (autofluorescence), IgG, β-amyloid and vascular endothelial growth factor (VEGF).

(ii) Oxidative damage and apoptosis Immunohistochemistry on frozen sections with antibodies to 8-hydroxy-2-deoxyguanosine (8-OHdG) and to acrolein will be used to detect oxidative damage and lipid peroxidation respectively. TUNEL assay will be used to detect apoptotic cell death.

(iii) ELISA assays for complements C3a and Ca5, Western blot analyses for HO-1, ABCA1 and Cyclin D1, and carbonyl protein adducts will be carried out in the different layers of the retina microdissected using LCM technology.

(3) Statistical analyses.

The data will be presented as mean values±SEM. Statistically significant differences between the groups will be analyzed using ANOVA followed by Tukey's post-hoc test for comparison of multiple treatments. Statistical analysis will be performed with GraphPad Prism software 4.01. A probability value of ≦0.05 is considered as significant.

D. Expected Outcomes.

Ccl-2^{-/-} mice exhibit vironectin, lipfuscin and Aβ deposits in drusen beneath the retinal pigment epithelium (RPE). Retina and Bruch's membrane thickening, photoreceptor atrophy, choroidal neovascularization, cell density reduction, complement and IgG deposition in RPE and VEGF production, oxidative damage, lipid peroxidation and apoptosis will also be present. It is believed that all or some of these pathological features will be prevented or reduced by the LXR antagonist, the ER agonist or the combination of the LXR antagonist and the ER agonist. Such results will demonstrate the contribution of each of the LXR and ER pathway in regulating proteins and morphological features relevant to AMD. It is believed that agents that inhibit LXRs and activate ERs can be used to treat or stop the progression of AMD. Both LXRs and ERs localize in retina and targeting these pathways may potentially represent a novel avenue for therapeutic strategies for AMD.

### DISCUSSION

Previously, it has been shown that the oxysterol 27-OHC reduces TH expression and induces an elevation in α-synuclein expression (J. P. Rantham Prabharkara et al., J. Neurochem., 107, 1722 (2008)). However the molecular mechanisms by which 27-OHC elicited these effects were not known. Reduction in TH and elevation in α-synuclein expression are important biochemical events in the pathogenesis of PD.

These examples demonstrate that ERβ, but not ERα, signaling positively regulates TH expression. 27-OHC reduces TH expression by exhibiting SERM activity and attenuating ERβ-induced TH expression, and this effect of 27-OHC is reversed by concomitant estradiol treatment. These examples further demonstrate that LXRβ, and not LXRα, signaling positively regulates α-synuclein expression. 27-OHC increases α-synuclein expression via the activation of LXRβ and this effect is reversed by concomitant treatment with the LXR antagonist ECHS.

27-OHC, an oxysterol with a steroid nucleus, exhibits selective estrogen receptor modulator (SERM) properties. Some studies have reported that 27-OHC activates ER, while others studies have found that 27-OHC acts as an ER-antagonist (Umetani et al., DuSell et al., cited above).

The general consensus is that 27-OHC and several other SERM (4-hydroxy tamoxifen) have tissue specific effects contingent on a multitude of coincident stimuli. There is a plethora of evidence suggesting that estradiol induces the expression of TH. For example, see L. Serova et al., Neuroendocrin., 75, 193 (2002). Furthermore, several studies have shown that the effects of estradiol on TH expression are mediated by ERα and ERβ. For example, see S. Marharjan et al., J. Neurochem., 112, 42 (2010). Several studies have characterized the neuroprotective role of estrogens in the preservation of dopaminergic cells in both in vivo and in vitro models of PD (H. Sawada et al., J. Neurosic. Res., 54, 707 (1998); S. Callier et al., Synapse, 37, 245 (2000); M. Grandbois et al., Neuroreport, 11, 343 (2000); S, Callier et al., Synapse, 41, 131 (2001)).

Estradiol, the most prominent endogenous estrogen and an agonist of ERα and ERβ, has been shown to increase TH immunoreactivity in dopaminergic neurons (V. Gayrard et al., Biol. Reprod., 50, 1168 (1994)). Estradiol increases TH promoter activity in ovariectomized TH9-LacZ transgenic mice, suggesting that the positive effect of estradiol on TH promoter activity is transcriptional in nature (N. R. Thanky et al., Brain Res. Mol. Brain Res., 104, 220 (2002)). The TH promoter contains a multitude of response elements for transcription factors that may regulate TH promoter activity in response to Estradiol including AP1, Sp1, and CREB (B. P. Fung et al., J. Neurochem., 58, 2044 (1992); A. Nakashima et al., Brain Res. Mol. Brain Res., 112, 61 (2003); E. J. Kilbourne et al., J. Biol. Chem., 267, 7563 (1992); K. S. Kim et al., J. Biol. Chem., 268, 15689 (1993); L. J. Lewis-Tuffin et al., Mol. Cell Neurosic., 25, 536 (2004)).

Furthermore, the TH promoter contains an Estrogen Response Element (ERE) half site to which activated estrogen receptors (ERα and ERβ) can bind and induce TH promoter activity (L. Serova et al., Neuroendocrin., 75, 193 (2002)).

The Examples above confirm that 27-OHC regulates TH expression via the modulation of ER. 27-OHC decreases ERα and ERβ protein levels in the nucleus. Furthermore, to correlate the decreased ERα/β levels with lower transcriptional activity, EMSA, ChIP and TH promoter analyses were performed to elucidate the role of ERα/β in the transcriptional regulation of TH expression and determine the extent to which 27-OHC affects TH transcription via the modulation of ERα/β.

The ERα and ERβ exhibit high homology and belong to the steroid receptor superfamily. However ERα and ERβ possess different transactivation domains that confer unique properties with respect to each other. The respective activation function (AF) motifs in ERα and ERβ can differentially interact with transcriptional co-activators, co-repressors, and co-integrators thereby bestowing ERα and ERβ with disparate biological activities and functions. EMSA analysis clearly showed that 27-OHC decreases ERα/β binding to the double stranded oligonucleotide probe that corresponds to the ERE-half site in the TH promoter.

The subtype of ER specifically involved in the regulation of basal and 27-OHC attenuation in TH expression was determined. ChIP analyses shows that, in the basal state, ERβ bound to the ERE-half site in the TH promoter is 10 times higher than ERα bound to the same site. Furthermore, 27-OHC attenuated the ERβ binding to the ERE half site in the TH promoter while having no effect on ERα binding to the same site.

This suggests that ERβ predominantly regulates basal expression of TH in SH SY5Y cells and 27-OHC-induced inhibition of TH expression is mediated via ERβ. Maharjan and colleagues have demonstrated in PC12 cells that estradiol treatment causes an increase in TH expression via ERα while eliciting attenuation in TH expression through ERβ (S. Maharjan et al. (2009) cited above). In this light, the results of these examples are novel and further substantiate a critical role of ERβ in the brain.

ERβ knock-out mice have markedly diminished cognitive functions (W. Krezel et al., PNAS USA, 98, 12278 (2001)). It is the general consensus that ERβ mediates the non-reproductive biological functions of estradiol in the brain (M. J. Weiser et al., Brain Res. Rev., 57, 309 (2008)). ERα and ERβ are pervasively expressed throughout the brain and spinal cord. Of relevance to PD, the substantia nigra almost exclusively expresses ERβ receptors (A. Quesada et al., J. Comp. Neurol., 503, 198 (2007)).

27-OHC is an endogenous ligand and activator of LXR (see B. Forman et al., PNAS USA, 94, 10588 (1997)). 27-OHC activates LXR-target genes in cultured HEK293 cells (X, Fu et al., J. Biol. Chem., 276, 38378 (2001)). The two receptor subtypes (namely LXRα and LXRβ) exhibit 77% homology, but have significantly different expression profiles (S. Alberti et al., Gene, 243, 93 (2000)). LXRα and LXRβ modulate transcription of target genes by forming heterodimers with Retinoid X Receptor α (RXRα) and subsequently binding to LXRE in the promoter regions of target genes. Using in silico analysis Cheng and colleagues (cited above) have identified functional LXRE in the human α-synuclein promoter region.

These examples demonstrate that 27-OHC increases α-synuclein expression levels. To determine if 27-OHC increases α-synuclein expression by activating LXRα/β and increasing the binding of LXRα/β to the LXRE in the α-synuclein promoter an EMSA was performed to determine the LXRα/β interaction with an exogenous consensus sequence corresponding to the LXRE in the α-synuclein promoter region. Increased binding of LXRα/β to this oligomeric probe was found, comprising the LXRE in the α-synuclein promoter. A ChIP assay was carried out to further characterize the involvement of LXR and elucidate the subtype of LXR that mediates 27-OHC-induced increase in α-synuclein expression. It was found that, in the basal state, LXRβ bound to the LXRE in the α-synuclein promoter region is 5.5-fold higher compared to LXRα. 27-OHC increased LXRβ binding to the LXRE in the α-synuclein promoter region, but produced no effect on LXRα binding to the same site. Furthermore, the LXR agonist GW3965 also increased LXRβ binding to the LXRE on the α-synuclein promoter without eliciting any increase in LXRα binding to the same site. The LXR antagonist ECHS on the other hand, decreased both the basal LXRβ binding as well as 27-OHC-induced increase in LXRβ binding to the LXRE on the α-synuclein promoter, but produced no effect on LXRα binding to the same site.

These results implicate LXRβ in the regulation of basal expression of α-synuclein as well as 27-OHC-induced increased α-synuclein expression.

LXRβ is the main LXR subtype in the brain (T. Kaino et al., J. Mol. Neurosci., 7, 29 (1996)). However these results must be tempered because there is no direct evidence for the subtype of LXR expressed in the dopaminergic neurons of the substantia nigra. Although, LXRα and LXRβ exhibit 77% homology and bind their endogenous ligands with relative similar affinity, their endogenous physiological functions, target genes and the effects on target genes may vary (K. Prufer et al., J. Cell Biochem., 100, 69 (2007)).

Recent evidence indeed suggests that LXRα and LXRβ differentially regulate target genes involved in lipid metabolism (J. Prufer, cited above). It is now the consensus that the canonical effects of LXRs, such as upregulation of ATP-binding cassette transporters ABCA1, ABCG1, ABCG5, ABCG8, upregulation of the expression of proteins such as ApoE and lipoprotein lipase involved in lipoprotein metabolism, and upregulation of the transcription factor SREBP-1c involved in the expression of lipogenic enzymes are mediated by LXRα, and not LXRβ. For example, see, J. J. Repa et al., Genes Dev., 14, 2819 (2000)).

Further corroboration of the differential effects of LXRα and LXRβ has surfaced from LXRα and LXRβ knock-out mice. Knock-out of LXRα in mice results in lower expression of lipogenic genes such as SCD-1 and FAS, while knock-out of LXRβ elicited no effect (S. Alberti et al., J. Clin. Invest., 107, 565 (2001)). Instead, LXRβ knock-out mice exhibited augmented expression of lipogenic enzyme ACC (S. Alberti et al., cited above) and the pivotal transcription factor SREBP-1c involved in the expression of lipogenic genes (G. U. Schuster et al., Circulation, 106, 1147 (2002). Therefore, inference can be drawn from afore mentioned studies that LXRα and LXRβ have diverse and opposite roles in lipid metabolism.

LXRβ represses the expression of lipogenic genes, while LXRα induces their expression. In light of this fact, lipogenic organs such as the liver and adipose tissue express LXRα more abundantly than LXRβ. Therefore the relative expression of LXRα and LXRβ in a given cell-type or tissue will determine the effects of endogenous LXR ligands (oxysterols) and synthetic LXR agonists (GW3965).

Consistent with this idea, LXR agonists designed pharmacologically to induce the beneficial effects of LXR on cholesterol metabolism also induce hepatic steatosis by increasing lipogenesis in the liver because of predomination of LXRα over LXRβ expression in the liver. In this study, we have demonstrated that the oxysterol 27-OHC and the synthetic LXR agonist increased the nuclear levels of both LXRα and LXRβ. However, both 27-OHC and GW3965 increased the binding of LXRβ, and not LXRα, to the α-synuclein promoter. This may be primarily attributed to the fact that α-synuclein may be an LXRβ regulated gene and not because LXRβ is more abundantly expressed in SH-SY5Y cells.

In summary, these results demonstrate that the oxysterol 27-OHC modulates expression of TH and α-synuclein via two distinct pathways. 27-OHC decreases TH expression by attenuating ERβ-mediated transcription of TH and increases α-synuclein by augmenting LXRβ-mediated transcription of α-synuclein. Attenuation of TH expression and elevation of α-synuclein expression are two important biochemical events implicated in PD. These results explicate the pathogenesis of sporadic PD since high levels of 27-OHC were found in the cortices of patients with PD and Lewy body dementia (D. A. Bosco et al., Nat. Chem. Biol., 2, 249 (2006)). Regulation of 27-OHC levels as well as concomitant activation of ER pathway and inhibition of LXR pathway are a target for the application of the therapeutic interventions disclosed and claimed herein to reduce the progression of PD and other neurodegenerative disorders of aging.

All publications, patents, and patent applications are incorporated herein by reference. While in the foregoing specification this present invention has been described in relation to certain preferred embodiments thereof, and many details have been set forth for purposes of illustration, it will be apparent to those skilled in the art that the present invention is susceptible to additional embodiments and that certain of the details described herein may be varied considerably without departing from the basic principles of the present invention.

What is claimed is:

1. A method of treating a mammal afflicted with an age-related disorder, the method comprising administering to the mammal a combination of liver X receptor (LXR) modulator and estrogen receptor (ER) modulator, in an amount effective to treat the mammal.

2. The method of claim 1, wherein the mammal is a human.

3. The method of claim 1, wherein the mammal is a human of at least about 50 years old in age.

4. The method of claim 1, wherein the age-related disorder comprises at least one of age-related macular degeneration (AMD), Parkinson's disease (PD), dementia with Lewy bodies (DLB), synucleinopathies, dyskinesias, (including bradykinesia, akinesia and dystonia), Alzheimer's disease (AD), dementia, multiple system atrophy (MSA), Shy-Drager syndrome, pure autonomic failure (PAF), and Pick disease (PiD).

5. The method of claim 1, wherein the age-related disorder comprises at least one of a cognitive dysfunction selected from the group consisting of dementia, age-related deficit in cognitive performance, stress-related deficit in cognitive performance, mild cognitive impairment (MCI), schizophrenia, Alzheimer's disease (AD), and symptoms thereof.

6. The method of claim 1, wherein the age-related disorder comprises at least one of dementia selected from the group consisting of vascular dementia (VaD), dementia of the Alzheimer's type, dementia due to HIV disease, dementia due to head trauma, dementia due to Parkinson's disease (PD), dementia due to Huntington's disease, dementia due to Pick's disease, dementia due to Creutzfeldt-Jacob disease, substance-induced persisting dementia, dementia due to multiple etiologies, dementia with Lewy bodies (DLB), ischemia/stroke, tangles, and global dementia.

7. The method of claim 1, wherein the age-related disorder comprises dementia of the Alzheimer's type selected from the group consisting of dementia of the Alzheimer's type without behavioral disturbance, dementia of the Alzheimer's type with behavior disturbance, dementia of the Alzheimer's type with early onset, and dementia of the Alzheimer's type with late onset.

8. The method of claim 1, wherein the age-related disorder comprises dry age-related macular degeneration (AMD) or wet age-related macular degeneration (AMD).

9. The method of claim 1, wherein the liver X receptor (LXR) modulator is a liver X receptor (LXR) antagonist.

10. The method of claim 1, wherein the liver X receptor (LXR) modulator is an α liver X receptor (LXRα) antagonist, or is a β liver X receptor (LXRβ) antagonist.

11. The method of claim 1, wherein the liver X receptor (LXR) modulator is an α liver X receptor (LXRα) modulator, or is a β liver X receptor (LXRβ) modulator.

12. The method of claim 1, wherein the liver X receptor (LXR) modulator comprises:
5α,6α-epoxycholesterol-3-sulfate (ECHS);
3-[3-[N-(2-Chloro-3-trifluoromethylbenzyl)-(2,2-diphenylethyl)amino]propyloxy]phenylacetic acid hydrochloride (GW3965);
ketocholesterol-3-sulfate;
2,4,6-Trimethyl-N-{[3′-(methylsulfonyl)-4-biphenylyl]methyl}-N-{[5-(trifluoromethyl)-2-furanyl]methyl}benzenesulfonamide (G-SK2033); or
5-choloro-N-T-n-pentylphenyl-1,3-dithiophthalimide (5CPPSS).

13. The method of claim 1, wherein the estrogen receptor (ER) modulator is an α estrogen receptor (ERα) modulator, or is a β estrogen receptor (ERβ) modulator.

14. The method of claim 1, wherein the estrogen receptor (ER) modulator is an α estrogen receptor (ERα) agonist, or is a β estrogen receptor (ERβ) agonist.

15. The method of claim 1, wherein the estrogen receptor (ER) modulator comprises
17β-estradiol (E2), Fulvestrant (ICI182780); Stilphostrol® (diethylstilbesterol diphosphate); or
Daidzein (7-Hydroxy-3-(4-hydroxyphenyl) chromen-4-one).

16. The method of claim 1, further comprising administering to the mammal at least one of an oxygenase inhibitor, a dopamine receptor agonist, dopamine precursor, monoamine oxidase B (MAO-B) inhibitor, catechol-O-methyltransferase (COMT) inhibitor, additional dopaminergic agent, anti-cholinergic, cholinesterase inhibitor, N-methyl-D-aspartic acid or N-methyl-D-aspartate (NMDA) receptor antagonist, and anti-psychotic (anti-depressant).

17. The method of claim 1, further comprising administering to the mammal the active pharmaceutical ingredient (API) of at least one of Requip® (ropinirole), Mirapex® (pramipexole), Parlodel® (bromocriptine), Apokyn® (apomorpine), Sinemet® (levodopa-carbidopa), Eldepryl® (selegiline-deprenyl), Emsam® (selegiline), Azilect® (rasagiline), Tasmar® (tolcapone), Comtan® (entacapone), Symmetrel® (amantadine), Artane® (trihexyphenidyl), Cogentin® (benzatropine), Aricept® (donepezil), Exelon® (rivastigmine), Namenda® (memantine), Clozaril® (clozapine), Abilify® (aripiprazole), and Zelapar® (selegiline hydrochloride), CERE-120, ACP-103 or SR57667B; wherein the active pharmaceutical ingredient (API) exists in a neutral form, as a free acid, as a free base, or as a pharmaceutically acceptable salt.

18. The method of claim 1, further comprising a pharmaceutically acceptable carrier or diluents.

19. The method of claim 1, wherein the administration is oral, parenteral, intravenous (i.v.), intraocular, intravitreal, intraperitoneal (i.p.) or stereotactic neurosurgical intracranial injection.

20. The method of claim 1, wherein the liver X receptor (LXR) modulator is administered in at least about 5 mg/day.

21. The method of claim 1, wherein the liver X receptor (LXR) modulator is administered once per day (q.d.), or twice a day (b.i.d.).

22. The method of claim 1, wherein the estrogen receptor (ER) modulator is administered in at least about 5 mg/day.

23. The method of claim 1, wherein the estrogen receptor (ER) modulator is administered once per day (q.d.), or twice a day (b.i.d.).

24. The method of claim 1, wherein the administration of the liver X receptor (LXR) modulator and the estrogen receptor (ER) modulator is approximately simultaneous or concurrent in time.

25. The method of claim 1, wherein the administration of the liver X receptor (LXR) modulator and the estrogen receptor (ER) modulator is consecutive in time.

26. The method of claim 1, wherein the liver X receptor (LXR) modulator and the estrogen receptor (ER) modulator are present within a single unit dosage form.

27. The method of claim 1, wherein the administration occurs for a period of time of at least about 4 weeks.


SEQUENCE LISTING (10 sequences)

SEQ ID NO: 1 - DNA, 20 bp, Artificial Sequence
  A synthetic primer
actggttcacggtggagttc

SEQ ID NO: 2 - DNA, 20 bp, Artificial Sequence
  A synthetic primer
agctcctgagcttgtccttg

SEQ ID NO: 3 - DNA, 20 bp, Artificial Sequence
  A synthetic primer
tgtgcccagtcatgacattt

SEQ ID NO: 4 - DNA, 20 bp, Artificial Sequence
  A synthetic primer
ccacaaaatccacagcacac

SEQ ID NO: 5 - DNA, 40 bp, Artificial Sequence
  A synthetic primer
gctttgacgtcagctcagcttataagaggctgctgggcca

SEQ ID NO: 6 - DNA, 30 bp, Artificial Sequence
  A synthetic primer
aagggaagcagatcataaaagttcagaaaa

SEQ ID NO: 7 - DNA, 18 bp, Artificial Sequence
  A synthetic primer
ctccatcaggcacagcag

SEQ ID NO: 8 - DNA, 18 bp, Artificial Sequence
  A synthetic primer
ggcccagcagcctcttat

SEQ ID NO: 9 - DNA, 20 bp, Artificial Sequence
  A synthetic primer
caagatgtgacttgggtgct

SEQ ID NO: 10 - DNA, 22 bp, Artificial Sequence
  A synthetic primer
tgggattttgttttctatcaca